(19) United States
(12) Patent Application Publication
(10) Pub. No.: US 2019/0178793 A1
(43) Pub. Date: Jun. 13, 2019

(54) CONTROLLED WATER ACTIVITY OF MICROORGANISMS FOR SPECTRAL IDENTIFICATION

(71) Applicant: THE ROYAL INSTITUTION FOR THE ADVANCEMENT OF LEARNING/MCGILL UNIVERSITY, Montreal (CA)

(72) Inventors: Ashraf A. ISMAIL, Westmount (CA); Pierre LEBEL, Saint-Laurent (CA); Jacqueline SEDMAN, Kirkland (CA)

(21) Appl. No.: 16/307,740

(22) PCT Filed: Jun. 6, 2017
(86) PCT No.: PCT/CA2017/050688

§ 371 Date: Dec. 6, 2018

Related U.S. Application Data
(60) Provisional application No. 62/346,087, filed on Jun. 6, 2016.

(51) Int. Cl. G01N 21/3563; C12Q 1/04; G01N 21/552; H01J 49/16; H01J 49/40

(52) U.S. Cl. CPC G01N 21/3563; C12Q 1/04; G01N 2021/3595; H01J 49/164; H01J 49/40; G01N 21/552

(57) ABSTRACT

The present disclosure presents methods and systems for the spectral identification of microorganisms based on controlled water activity. The water content of a sample is fully retained and no drying treatments are applied prior to spectral acquisition. Spectral acquisition of the sample is immediately preceded by a measurement of a water vapor level in the ambient atmosphere around the sample through a background spectrum. The spectral data and the background spectrum are combined and the combined data is used for analysis.

### CROSS-REFERENCE TO RELATED APPLICATIONS

The present application claims the benefit of U.S. Provisional Patent Application No. 62/346,087 filed on Jun. 6, 2016, the contents of which are hereby incorporated in their entirety by reference.

### TECHNICAL FIELD

The present disclosure relates generally to identifying microorganisms using spectral data obtained from infrared spectroscopy, and particularly to controlling the water activity of a sample for the purpose of spectral analysis.

### BACKGROUND OF THE ART

The use of infrared spectroscopy for microbial differentiation and identification dates back to 1954. The feasibility of such application of infrared spectroscopy was substantially enhanced by the advent of Fourier transform infrared (FTIR) spectroscopy and has been extensively investigated by numerous research groups over the past three decades. Taken together, this body of research indicates that the infrared spectra of pure microbial colonies serve as whole-organism fingerprints that are specific down to the subspecies level of taxonomic classification. However, the reliability of infrared spectroscopy as a means of microbial identification is dependent upon all the conditions employed in the identification procedure, beginning with growth of the microorganisms on culture media to obtain pure colonies and followed by sample preparation for infrared spectroscopic measurement, which entails the deposition of microbial cells, taken from one or more pure colonies, as a thin film on a suitable substrate.

While standardization of growth conditions may be achieved, the effects of variability in sample deposition and/or preparation may present certain challenges. For example, because water is a strong absorber of infrared light. samples are typically dried prior to spectral acquisition to avoid spectral interferences from water and to improve the signal-to-noise ratio of the spectral data. However, drying treatments have significant irreversible effects on the microorganism at the molecular level, owing to disruption of hydrogen bonding between polysaccharides and water, partial or complete denaturation of proteins, and alteration of the cellular membrane of the microorganism. In addition, the drying process adds significant time to the analysis, thus delaying results. And finally, it is extremely difficult to ensure reproducibility between samples when applying the drying treatments.

### SUMMARY

The present disclosure presents methods and systems for the spectral identification of microorganisms based on controlled water activity. The water content of a sample is fully retained and no drying treatments are applied prior to spectral acquisition. Spectral acquisition of the sample is immediately preceded by a measurement of a water vapor level in the ambient atmosphere around the sample through a background spectrum. The spectral data and the background spectrum are combined and the combined data is used for analysis.

In accordance with a first broad aspect, there is provided a method for spectral identification of a microorganism. The method comprises acquiring a background spectrum to measure a water vapor level of an ambient atmosphere; bringing a sample containing the microorganism into contact with an internal reflection element in such a manner that water content of the microorganism is fully retained; acquiring spectral data from the sample no more than a predetermined time after having acquired the background spectrum; combining the background spectrum and the spectral data, thereby producing modified spectral data; and characterizing the microorganism using the modified spectral data.

In some embodiments, the internal reflection element is a single-bounce attenuated total reflectance (ATR) element. In some embodiments, the single-bounce ATR element is a diamond ATR crystal.

In some embodiments, the background spectrum is acquired in a path between an infrared source and an infrared detector defined for acquisition of the spectral data while the internal reflection element is without the sample. In some embodiments, an interferometer is in the path between the infrared source and the infrared detector. In some embodiments, a linear variable filter is in the path between the infrared source and the infrared detector.

In some embodiments, the predetermined time is less than or equal to 60 seconds. In some embodiments, the predetermined time is comprised in a range of about two minutes to about five seconds.

In some embodiments, the sample is from a blood culture medium. In some embodiments, the sample is from a microbial culture. In some embodiments, the sample is from a clinical specimen.

In some embodiments, bringing the sample containing the microorganism into contact with the internal reflection element comprises placing the sample on a surface of the internal reflection element. In some embodiments, bringing the sample containing the microorganism into contact with the internal reflection element comprises providing the sample in a support that acts as a reservoir to retain the water content of the microorganism.

In some embodiments, the method further comprises comparing the water vapor level of the modified spectral data to a first threshold and rejecting the modified spectral data when the water vapor level is above the first threshold.

In some embodiments, the method further comprises comparing the water content level of the modified spectral data to a second threshold and rejecting the modified spectral data when the water content level is below the second threshold.

In some embodiments, the method further comprises comparing a biomass of the sample, extracted from the modified spectral data, to a third threshold and rejecting the modified spectral data when the biomass is below the third threshold.

In some embodiments, characterizing the microorganism comprises comparing the modified spectral data to reference data to determine an identity of the microorganism.

In some embodiments, the reference data is constructed from known microorganisms obtained from two or more culture media using data points common to reference spectral data obtained from the two or more culture media.

In some embodiments, acquiring spectral data from the sample comprises acquiring an attenuated total reflectance Fourier transform infrared spectrum.

In some embodiments, the method further comprises using the modified spectral data to enhance the characterization of the microorganism by matrix-assisted laser desorption/ionization time-of-flight mass spectrometry.

In accordance with another broad aspect, there is provided a system for spectral identification of a microorganism. The system comprises a processing unit and a non-transitory computer-readable memory communicatively coupled to the processing unit and comprising computer-readable program instructions. The program instructions are executable by the processing unit for receiving a background spectrum comprising a water vapor level of an ambient atmosphere; receiving spectral data from a sample containing the microorganism and acquired no more than a predetermined time after the background spectrum is acquired, the microorganism in the sample having an intact water content; combining the background spectrum and the spectral data, thereby producing modified spectral data; and characterizing the microorganism using the modified spectral data.

In some embodiments, the predetermined time is less than or equal to 60 seconds. In some embodiments, the predetermined time is comprised in a range of about two minutes to about five seconds.

In some embodiments, the program instructions are further executable for comparing the water vapor level of the modified spectral data to a first threshold and rejecting the modified spectral data when the water vapor level is above the first threshold.

In some embodiments, the program instructions are further executable for comparing the water content level of the modified spectral data to a second threshold and rejecting the modified spectral data when the water content level is below the second threshold.

In some embodiments, the program instructions are further executable for comparing a biomass of the sample, extracted from the modified spectral data, to a third threshold and rejecting the modified spectral data when the biomass is below the third threshold.

In some embodiments, characterizing the microorganism comprises comparing the modified spectral data to reference data to determine an identity of the microorganism.

In some embodiments, the reference data is constructed from known microorganisms obtained from two or more culture media using data points common to reference spectral data obtained from the two or more culture media.

In some embodiments, the program instructions are executable for performing attenuated total reflectance Fourier transform infrared spectroscopy.

In some embodiments, the program instructions are further executable for using the modified spectral data to enhance the characterization of the microorganism by matrix-assisted laser desorption/ionization time-of-flight mass spectrometry.

In accordance with a further broad aspect, there is provided a non-transitory computer readable medium having stored thereon program code executable by a processor for spectral identification of a microorganism. The program code is executable for receiving a background spectrum comprising a water vapor level of an ambient atmosphere; receiving spectral data from a sample containing the microorganism and acquired no more than a predetermined time after the background spectrum is acquired, the microorganism in the sample having an intact water content; combining the background spectrum and the spectral data, thereby producing modified spectral data; and characterizing the microorganism using the modified spectral data.

### BRIEF DESCRIPTION OF THE DRAWINGS

Further features and advantages of the present invention will become apparent from the following detailed description, taken in combination with the appended drawings, in which:

FIGS. 1A and 1B are example setups for acquiring spectral data from a sample;

FIG. 2 is a flowchart of an example method for spectral identification of microorganisms;

FIG. 3 is an example of a background spectrum;

FIG. 4 is another example of a background spectrum;

FIG. 5 is an enlarged view of a region of the background spectrum of FIG. 4;

FIG. 6 is an example of a multi-tier structure for classification;

FIG. 7 is a breakdown of the multi-tier structure of FIG. 6, in accordance with one embodiment;

FIG. 8 is four example sets of spectral data shown separately;

FIG. 9 shows the four sets of spectral data from FIG. 8 in an overlaid fashion;

FIG. 10 is an example of modified spectral data with a region of lower noise;

FIG. 11 is an example of modified spectral data with validation regions;

FIG. 12 is an example of modified spectral data that is non-compliant with regards to water vapor and sample water content;

FIG. 13 is an example of modified spectral data that is non-compliant with regards to sample biomass;

FIG. 14 illustrates an alternative manner of validating sample biomass;

FIG. 15 illustrates an example database structure having multiple tiers;

FIGS. 16A-16F illustrate discriminating between Gram-positive and Gram-negative bacteria, genera, species, and strains based on differences in their spectra;

FIG. 17 illustrates clustering of MRSA and MSSA clinical isolates based on differences in their spectra;

FIG. 18 illustrates an example differentiation between Enterococcus faecalis and E. faecium using ATR-FTIR spectroscopy followed by MALDI-TOF MS;

FIG. 19 is an example system for spectral identification of microorganisms;

FIG. 20 is an embodiment for a microorganism identification device;

FIG. 21 is an embodiment for an application running of the microorganism identification device of FIG. 20;

FIG. 22 illustrates an example contrast between using an average spectrum and using spectral regions selected by implementing a feature selection algorithm;

FIGS. 23A-23D are examples of differentiation between various microorganisms;

FIGS. 24A-24E are further examples of differentiation between various microorganisms;

FIG. 25A is an example of discriminating among yeast species;

FIG. 25B is an example of differentiation between vanA and vanB genotypes of vancomycin-resistant Enterococcus faecium;

FIG. 25C is an example of differentiation among methicillin-sensitive Staphylococcus aureus (MSSA) and three Canadian epidemic clones of methicillin-resistant S. aureus (CMRSA-2, CMRSA-7, and CMRSA-10); and

FIGS. 26A-26B illustrate an example where ATR-FTIR spectroscopy was used to enhance the differentiation between E. coli and Shigella species by MALDI-TOF MS.

It will be noted that throughout the appended drawings, like features are identified by like reference numerals.

### DETAILED DESCRIPTION

There are described herein methods and systems for spectral identification of a microorganism. The microorganism may be any microscopic living organism that is single-celled, such as but not limited to bacteria, archaea, yeasts, fungi, and molds. A sample of the microorganism is provided on an internal reflection element. The sample may be solid or liquid, and contains intact microbial cells having an intact water content level. An intact water content level should be understood to mean that no drying treatments are applied to the sample, and no reagents are used to reduce or eliminate the original water content of the sample. Drying leads to variability in the spectral profile that may be acquired from any given microorganism, and thus has a negative impact on the reliability of identification of the microorganism.

Spectral identification is thus performed based on characteristic spectral fingerprints of intact, whole organisms, with minimal post-culture sample preparation required. Spectral databases of well-characterized strains and multivariate statistical analysis techniques are used to identify unknowns by matching their spectra against those in the database.

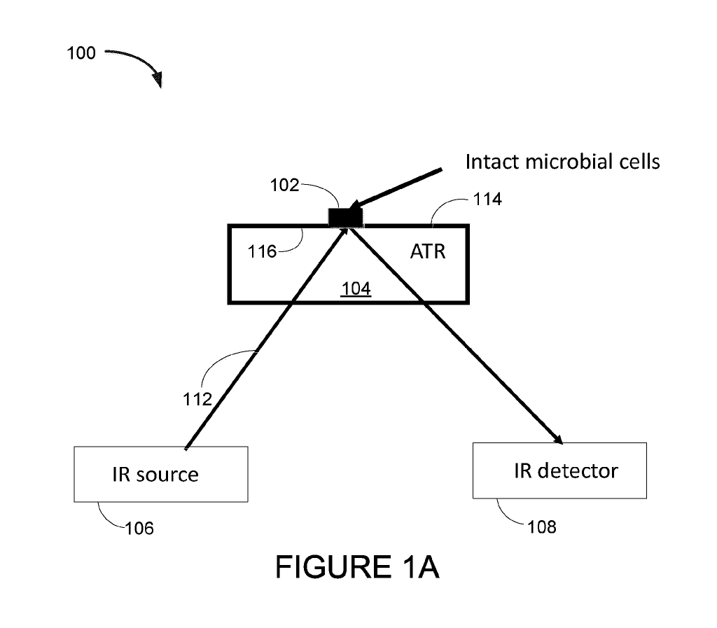

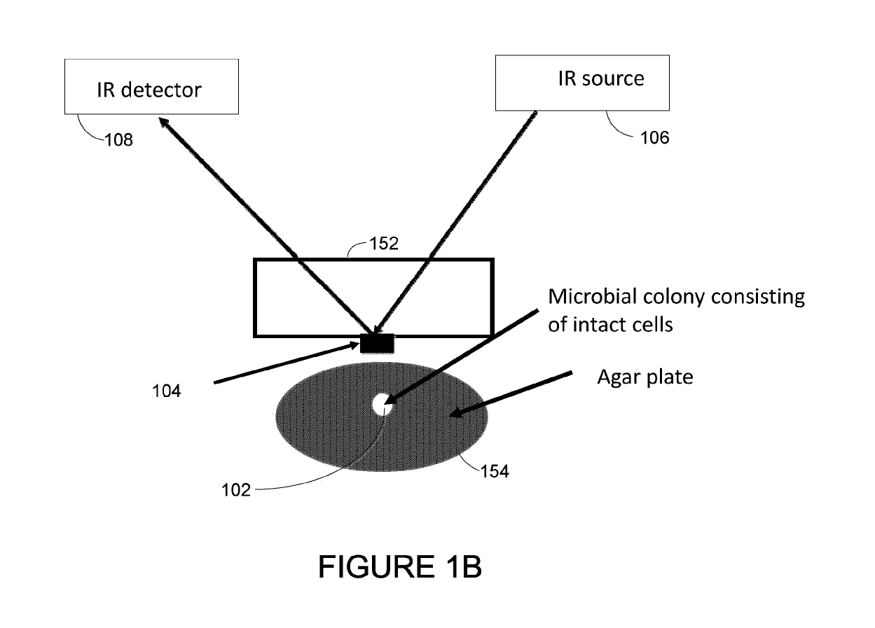

FIG. 1A illustrates an example setup 100 used for spectral identification of a microorganism. The sample 102 sits on a surface 114 of an internal reflection element 104. The sample 102 may be taken from any known culture medium without breaking the culture medium surface and deposited onto the internal reflection element 104 using a transfer device (not shown) such as a sterile toothpick or loop. Alternatively, the sample may be placed into contact with the surface 114 of the internal reflection element 104 without transferring the sample from the culture medium. Any setup whereby the sample 102 is in contact with the surface 114 of the internal reflection element 104 while retaining the water content of the microorganism may be used. Note that while FIG. 1A illustrates the sample 102 as being on top of the element 104, the setup may be inverted such that surface 114 is a bottom surface of the element 104, and sample 102 is on the bottom of the element 104. The sample 102 may be lowered or raised in order to contact the surface 104, using manual or automatic means. Alternatively, the element 104 may be lowered or raised in order to contact the sample 102, using manual or automatic means. An example is illustrated in FIG. 1B, whereby a device support 152 holding the internal reflection element 104 is lowered in order to contact the sample 102 held in a sample support 154, such as an agar plate.

The sample 102 may be obtained from a microbial culture, a blood culture, bodily fluids (such as urine and pus, nasal and wound swabs), food, water, air, and the like. The size of the sample 102 should be sufficient to cover an area of the internal reflection element 104. In some embodiments, the sample 102 covers a majority of the surface 114 of the element 104. Alternatively, the sample 102 covers less than half of the surface 114. In some embodiments, the sample 102 is sized to be about one to two millimeters in diameter. Other sample sizes may also be used. Any thickness of the sample 102 may be used.

The internal reflection element 104 is made of an optical material having a higher refractive index than the sample 102, so that reflection of a beam 112 off an internal surface 116 in contact with the sample 102 at an angle that exceeds the critical angle creates an evanescent wave (not shown) which extends into the sample 102. In some embodiments, the internal reflection element 104 is an attenuated total reflectance (ATR) crystal, and in some embodiments a diamond ATR crystal. Other materials, such as zinc selenide, may also be used for the internal reflection element 104. A beam 112 of infrared light is passed through the internal reflection element 104 in such a way that it reflects at least once off the internal surface 116 in contact with the sample 102. The internal reflection element 104 may thus be a single-bounce ATR crystal or a multi-bounce ATR crystal. Various optical components, such as lenses and/or mirrors, may be used to direct the beam 112 from a light source 106 to the internal reflection element 104 and back towards a detector 108.

In some embodiments, the internal reflection element 104 is mounted inside an infrared spectrometer, which may be a Fourier transform infrared (FTIR) spectrometer or a dispersive spectrometer. Any device that can acquire an infrared spectrum in the spectral region between 4000 and 400 wavenumbers and that can be coupled with an attenuated total reflectance accessory, such as devices that are filter-based, variable filter array-based, FTIR-based, and quantum cascade laser (QCL)-based, may be used. The light source 106 may be an infrared light source configured to emit light at one or more wavelengths, and the detector 108 may be an infrared detector configured for detecting the reflected beam 112 at a single detection point or a plurality of detection points corresponding to different regions of the sample 102. In some embodiments, the infrared spectrometer is an FTIR spectrometer operating in rapid-scan mode and having an infrared microscope and a focal-plane-array (FPA) detector, such as a 32×32 array of detector elements, referred to herein as an FPA-FTIR spectrometer. In some embodiments, the infrared spectrometer is a dispersive spectrometer that employs a linear variable filter and a pyroelectric detector array.

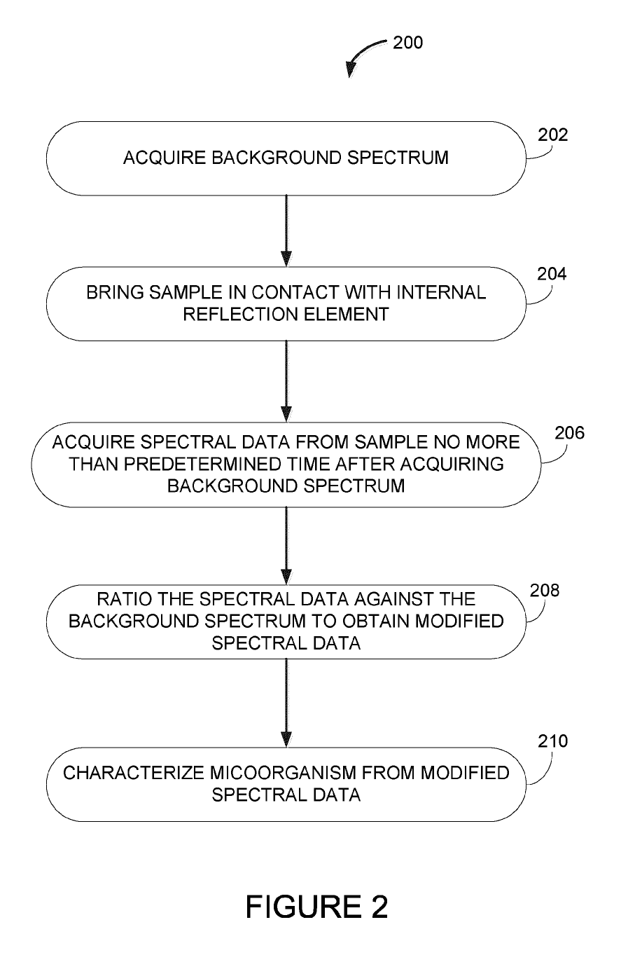

Referring to FIG. 2, there is illustrated a method 200 for identification of a microorganism using the setup 100 of FIG. 1A. At step 202, a background spectrum is acquired. The background spectrum measures a water vapor level of the ambient atmosphere in the path between the light source 106 and the detector 108. For example, the beam 112 may be measured by the detector 108 when the surface 114 of the internal reflection element 104 is without the sample.

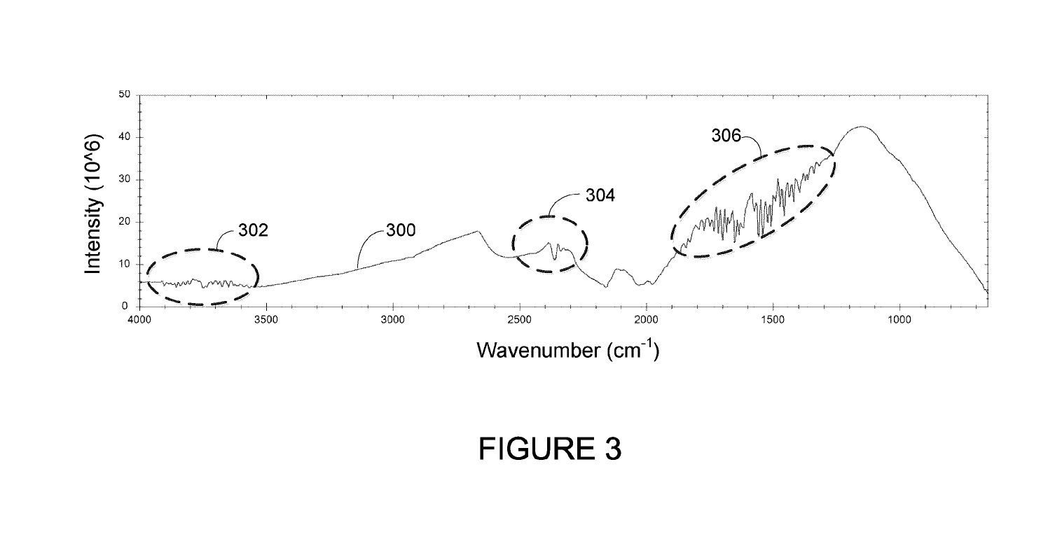

FIG. 3 is an example of a background spectrum 300. The region 302 of the background spectrum 300 is representative of the water vapor in the atmosphere. The region 304 is representative of the CO₂ in the atmosphere. The region 306 is representative of the water vapor in the atmosphere. The signal 300 was acquired by co-adding 64 scans taken during 45 seconds. Note that fewer scans, such as 4, 16, and 32, may be used, and more scans, such as 128 and 256, may be used.

Referring back to FIG. 2, once the background spectrum has been acquired, as per step 202, the sample 102 is brought into contact with the internal reflection element 104 using any automated and/or manual means in a manner to fully retain the water content of the intact microbial cells comprising or contained within the sample, as per step 204. As explained above, the sample 102 may be transferred onto the internal reflection element 104 using any type of transfer device. Alternatively, the sample may be brought into contact with the internal reflection element 104 while remaining inside a support. For example, the support may be the culture medium, such as an agar culture plate or another growth medium. In some embodiments, the support acts as a reservoir of moisture to ensure that the water content level of the microorganism is fully retained. In some embodiments, evaporation of the water from the sample is prevented by a physical means.

At step 206, spectral data from the sample is acquired no more than a predetermined amount of time after bringing the sample 102 into contact with the internal reflection element 104 whereby the water content of the intact microbial cells comprising or contained within the sample is fully retained. In some embodiments, the predetermined amount of time is less than or equal to one minute. In some embodiments, the predetermined amount of time is selected from a range of about two minutes to about five seconds. In some embodiments, the predetermined amount of time is the minimal time it takes to swab the culture medium, apply the sample to the element 104, and press scan on the spectrometer. When automated, the sample 102 may be kept at a very close distance to the element 104 without being in contact therewith while the background spectrum is acquired, followed by immediate contact of the sample 102 with the element 104 and acquisition of the spectral data. A full spectral range from 4000 cm⁻¹ to 400 cm⁻¹ may be acquired, even though spectral data from one or more narrower spectral regions may be employed for the purpose of enhancing reproducibility and accuracy of bacterial differentiation. In some embodiments, if it is desired to access spectral regions masked by H₂O absorption, for example, the spectral region between 1700 and 1600 cm⁻¹, the H₂O in the sample may be replaced by deuterium oxide (D₂O).

At step 208, the background spectrum and the spectral data are combined to obtain the modified spectral data. Combining the background spectrum and the spectral data may also be viewed as performing a ratio of the spectral data against the background spectrum. The acquisitions are combined to obtain a transmittance spectrum that is then used to produce an absorbance spectrum “A”. The time between the two acquisitions, namely of the background spectrum and the spectral data from the sample, is limited in order to prevent evaporation of the water content from the sample, and to ensure as close a match as possible of the water content of the sample with the water vapor found in the ambient atmosphere. As such, when the background spectrum and the spectral data are combined, water vapor bands are effectively eliminated from the spectral data.

In some embodiments, combining the background spectrum and the spectral data comprises dividing the sample data by the background data (to obtain the transmittance spectrum) and taking a logarithm of the result (to obtain the absorbance spectrum):

A=−log₁₀(sample/background)

The result (“A”) may be viewed as modified spectral data, as the water vapor bands from the sample spectral data have been removed, and it forms the basis of the analysis performed in order to characterize the microorganism, as per step 210.

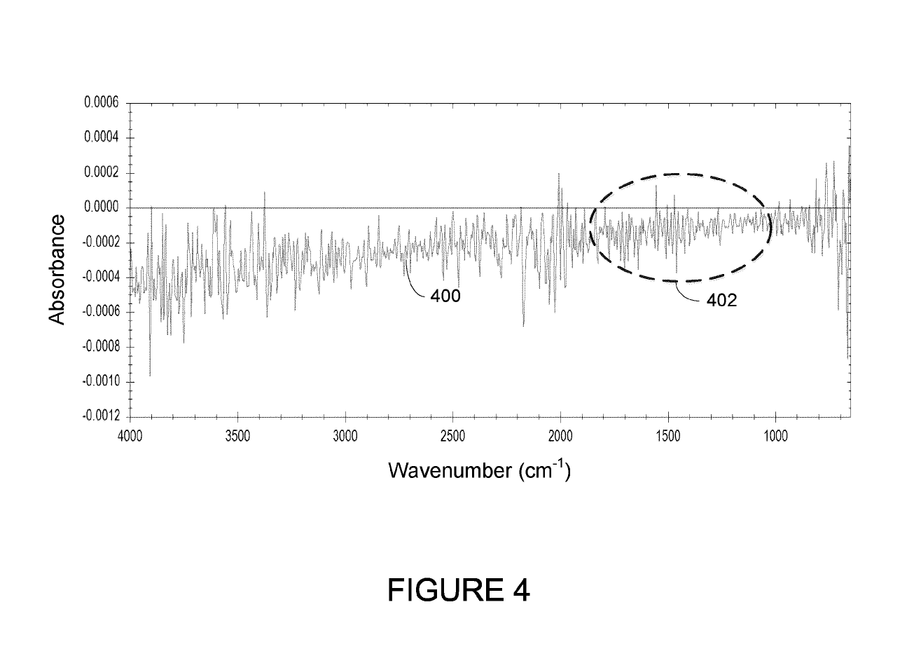

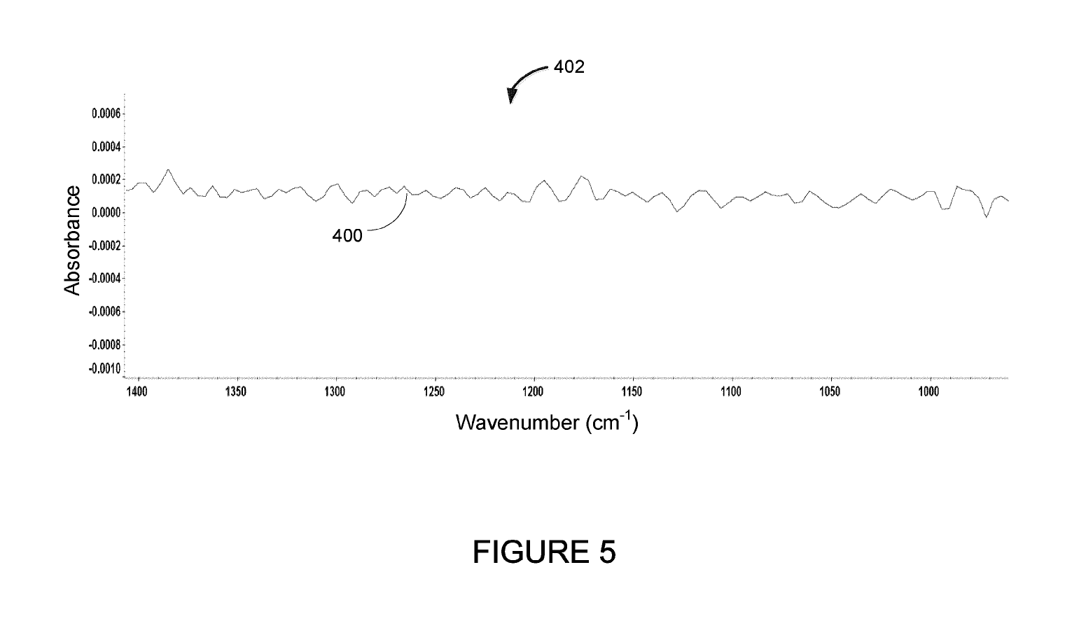

FIG. 4 is an example of modified spectral data acquired in the absence of a sample. The region 402 shows a peak-to-peak noise level of less than 0.0005 absorbance units. Note the absence of water vapor bands across the entire spectrum. FIG. 5 is an enlarged view of the spectrum 400 inside region 402. The peak-to-peak noise level is 0.0002 absorbance units for the range of 1406.765 cm⁻¹ to 957.953 cm⁻¹. The root-mean-square (RMS) noise level is 3.952*10⁻⁵.

In some embodiments, step 210 of the method 200 is performed as described in U.S. Pat. No. 9,551,654, the contents of which are incorporated by reference. For example, at least one multi-pixel spectral image of the sample is obtained, wherein each pixel of the image has a corresponding spectrum, and one or more spectra is selected from the spectral image based on one or more spectral characteristics of the corresponding spectrum. The microorganism may be identified by comparing the one or more selected spectra with spectra of reference microorganisms from a database. The modified spectral data is compared to those in the spectral databases containing spectra of pre-characterized isolates. Single or multiple multivariate methods may be employed for the identification of the isolate. Among the multivariate methods are hierarchical cluster analysis (HCA), principal component analysis (PCA), partial least squares (PLS), and spectral search which generate a similarity match between the spectra of unknown isolate and a near identical spectrum in the spectral database. It should be noted that selected spectral regions rather than the full spectrum may be employed in the identification procedure.

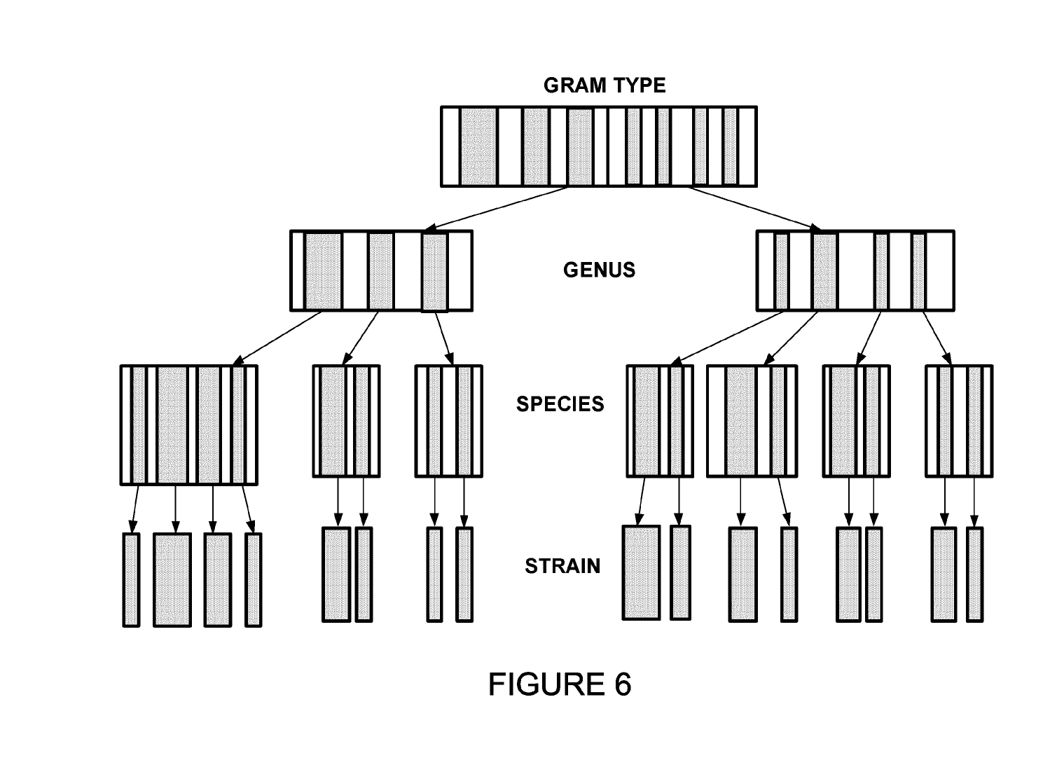

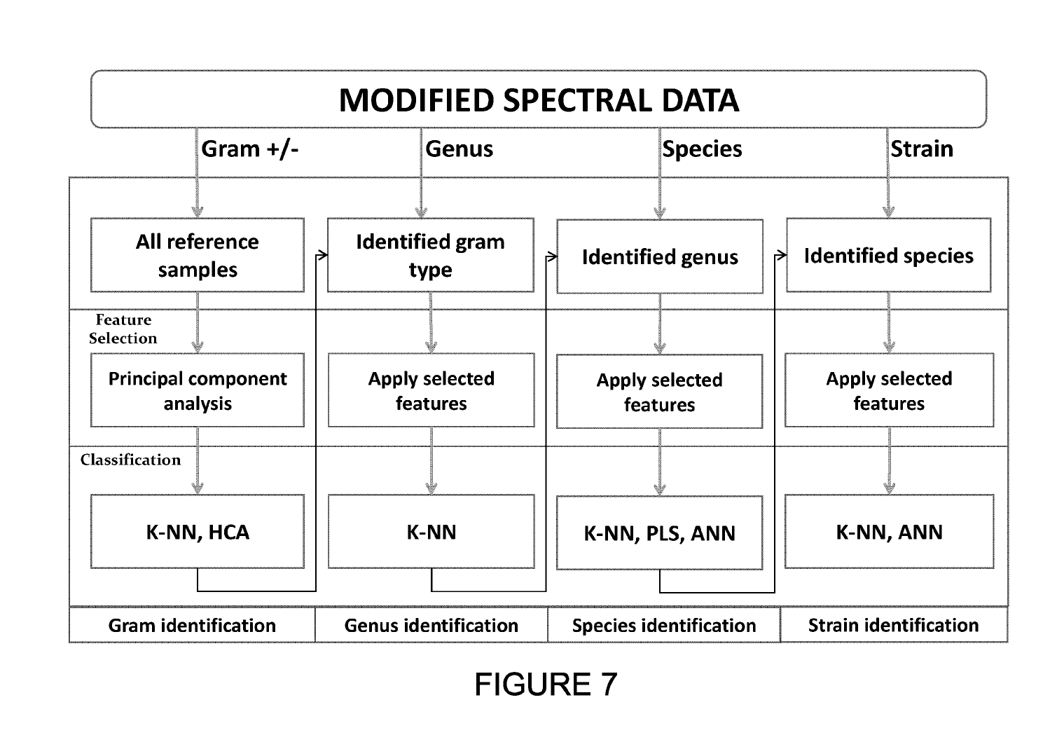

As illustrated in FIG. 6, a multi-tier classification strategy may be used, and classification may be performed at each taxonomic level. Classification models may be developed using appropriate subsets of the spectra in the database as training sets. Each classification model may be optimized using a feature selection algorithm to identify the spectral features that best characterize the desired classification. The shaded regions in FIG. 6 denote selected regions for the feature selection algorithm. The microorganism may thus be classified in accordance with Gram-stain type (i.e. positive or negative), genus, species, and strain, as per FIG. 7. A microorganism may further be determined to be an antibiotic-resistant strain or an antibiotic-sensitive strain. For each level of classification, analysis may be employed to find spectral features that differentiate between types. For example, specific spectral regions within the ATR-FTIR spectrum may be selected to separate the Gram-positive from the Gram-negative bacteria, this followed by a tier-wise separation at the genus, species, strain, and serotype levels and in some cases separation between antibiotic-resistant and antibiotic-sensitive strains and in some cases separation between genotypes. In some cases, toxin-producing bacteria can further be classified by the type of toxin they produce.

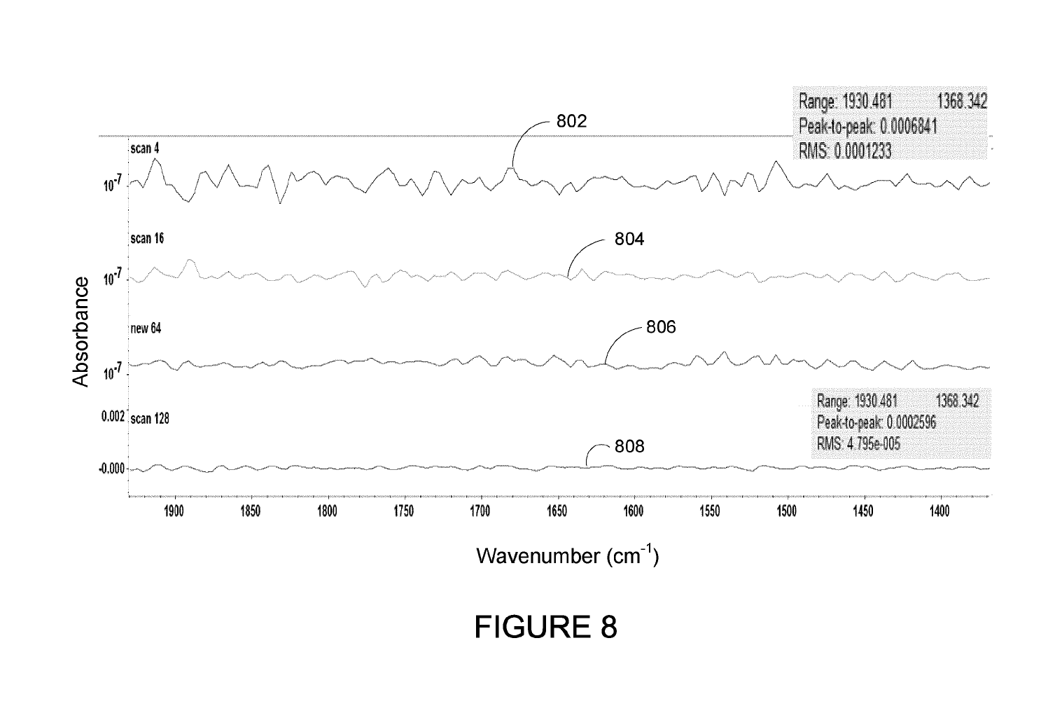

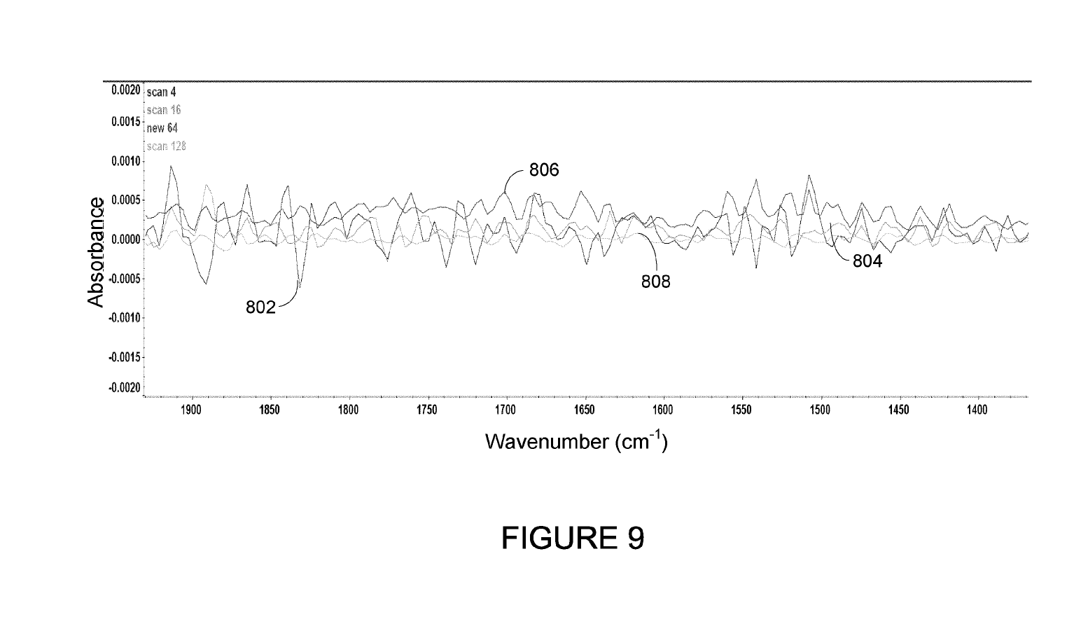

The signal-to-noise ratio (SNR) of the spectral data may be improved by performing a greater number of scans of the sample, such as 64, 128, or 256 instead of 4, 16, or 32. However, a greater number of scans means a longer scan time, increasing the difference between the water vapor in the background spectrum and the spectral data. The method 200 is thus a compromise: obtaining an acceptable SNR while minimizing the difference in water vapor level between the background spectrum and the spectral data. FIGS. 8 and 9 illustrate four signals 802, 804, 806, 808 taken from a blank with four different numbers of scans. In FIG. 8 the signals 802, 804, 806, 808 are shown separately while in FIG. 9 they are overlaid one on top of the other. Signal 802 was taken with 4 scans, signal 804 was taken with 16 scans, signal 806 was taken with 64 scans, and signal 808 was taken with 128 scans. The peak-to-peak noise level for the lowest number of scans, namely signal 802, is 0.0007 absorbance units. The peak-to-peak noise level for the highest number of scans, namely signal 808, is 0.0003 absorbance units. In some embodiments, the selected number of scans for the acquisition of the spectral data is 64. Other numbers of scans may also be used.

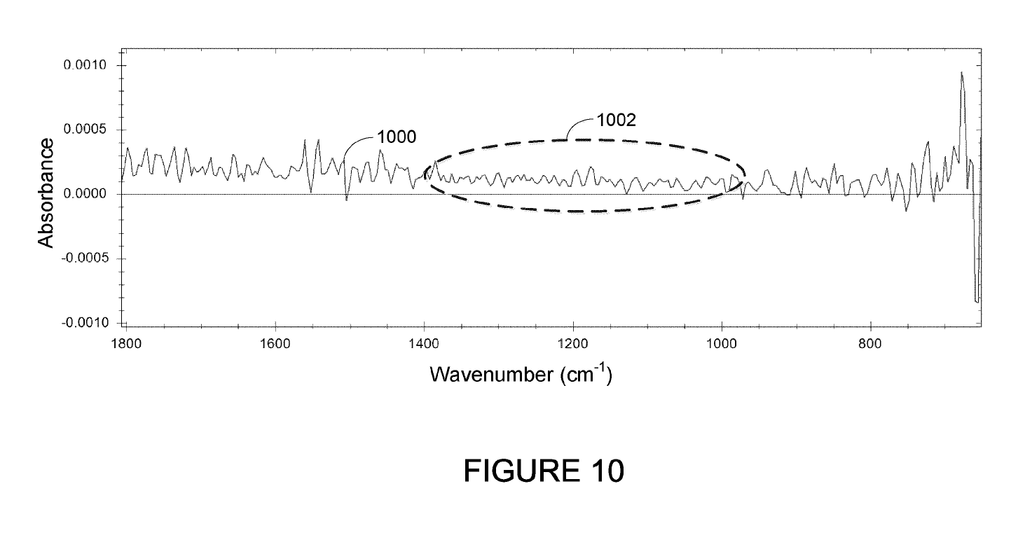

In some embodiments, a particular region of the data, which shows a suitable SNR, is used for the analysis. FIG. 10 illustrates an example, whereby region 1002 of the signal 1000 is found to show a lower noise level than the rest of the spectrum. In some embodiments, the data selected for analysis from the modified spectral data is taken from a range of about 1480 cm⁻¹ to about 980 cm⁻¹. In some embodiments, the range is about 1500 cm⁻¹ to about 1000 cm⁻¹. In some embodiments, the range is about 1760 cm⁻¹ to about 960 cm⁻¹. Other ranges may also be used.

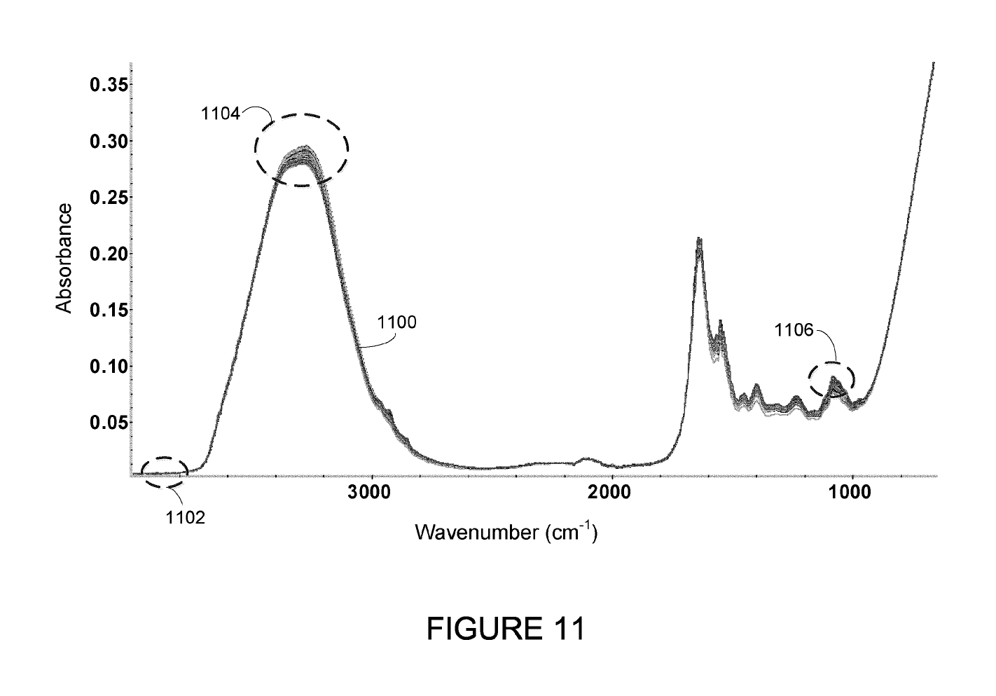

In some embodiments, the modified spectral data is validated with regards to various parameters, such as water vapor level, water content of the sample, and/or biomass of the sample. FIG. 11 is an example of the modified spectral data 1100. Region 1102 is used to validate water vapor level, region 1104 is used to validate sample water content, and region 1106 is used to validate biomass. The measurements obtained in each one of the regions 1102, 1104, 1106 may be compared with one or more corresponding threshold and/or range in order to validate each one of the parameters.

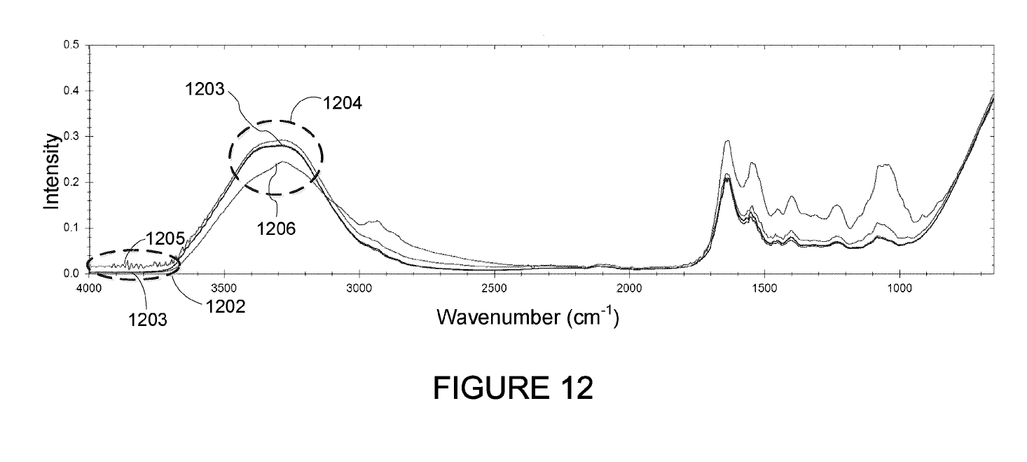

For example, the measurements of region 1102 may be compared to a first threshold. A measurement for water vapor level is considered acceptable if it falls below the first threshold, so as to ensure absence of spectral interference from water vapor in the modified spectral data. An example value for the first threshold is a peak-to-peak noise level in region 1102 of 0.001 absorbance units. Measurements above the first threshold are indicative of the presence of water vapor bands in the modified spectral data. The modified spectral data may be rejected as being non-compliant in such a case. FIG. 12 shows an example of a set of modified spectral data that is non-compliant with regards to water vapor level, as shown in region 1202. Signal 1205 is above the threshold, represented by signal 1203 and which may be a reference signal or a previously captured signal that is compliant. Therefore, there is spectral interference from water vapor in the modified spectral data. Validation may be performed visually by comparing the captured signal, such as 1205 to another signal, such as 1203 deemed compliant, or it may be performed automatically by comparing the measured values to a threshold value.

In another example, the measurements of region 1104 are compared to a second threshold. A measurement for water content of the sample is considered compliant if it is above the second threshold, so as to ensure that the water content of the sample is retained at the time of spectral acquisition. Values for the second threshold are dependent on the type of internal reflection element that is being employed. In embodiments in which the internal reflection element is a single-bounce diamond ATR crystal, an example value for the second threshold is signal intensity in region 1104 of 0.26 absorbance units. Measurements below the second threshold are indicative of a sample that has not retained its water content. The modified spectral data may be rejected as being non-compliant in such a case. Region 1204 in FIG. 12 shows an example of a set of modified spectral data that is also non-compliant with regards to the water content of the sample. Signal 1206 is shown to be significantly lower than signal 1203, which is used as the reference for the second threshold. Note that while the first threshold and second threshold are illustrated using the same reference signal 1203, separate signals may also be used. Validation may be performed visually by comparing the captured signal, such as 1206 to another signal, such as 1203, or it may be performed automatically by comparing the measured values to a threshold value.

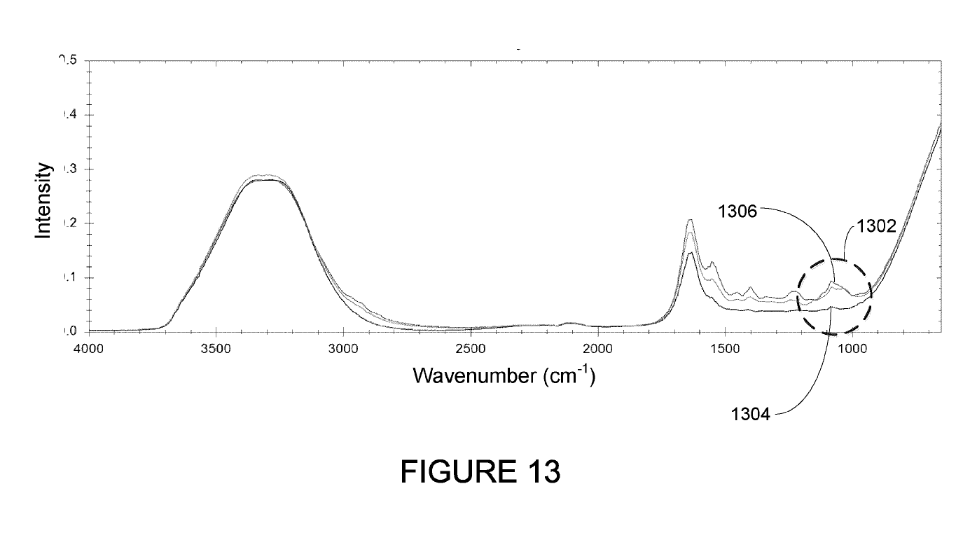

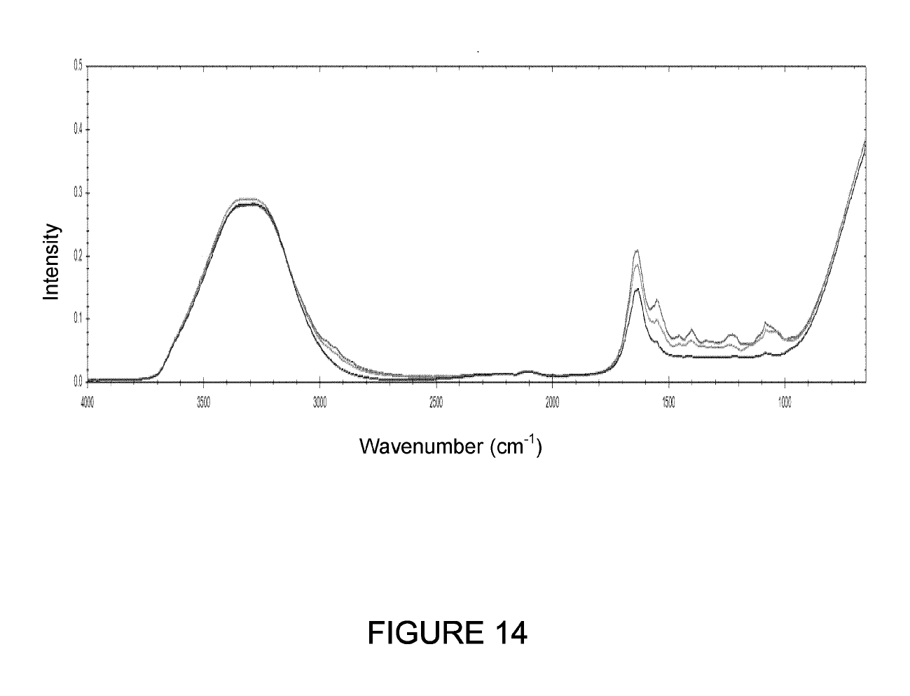

In some embodiments, biomass of the sample is validated in a similar manner as that shown with respect to water vapor level and sample water content. For example, in FIG. 13, region 1302 shows a signal 1304 that is lower than a reference signal 1306, which represents a third threshold for biomass. This is indicative of a biomass that is too low and therefore cause for rejection of the spectral data. Values for the third threshold are dependent on the type of internal reflection element that is being employed. In embodiments in which the internal reflection element is a single-bounce diamond ATR crystal, an example value for the third threshold is signal intensity in region 1302 of 0.010 absorbance units. Alternatively, or in combination therewith, the biomass of the sample may be evaluated by changes in the height of the amide II band of proteins, as illustrated in FIG. 14.

In some embodiments, in the event that the biomass is low relative to the free water within the sample, the free water is reduced by a number of techniques, such as allowing controlled evaporation of water, for example, by placing a desiccant over the sample (without contact with the sample), or warming the sample (e.g. with a use of a heat lamp placed above the sample, and or heating the ATR surface) or allowing the sample to be exposed to ambient conditions and acquiring multiple spectra as a function of time until the desired biomass/water ratio is achieved. This ratio can be determined by dividing the absorbance attributed to the bacteria at 1080 cm⁻¹ over the absorbance of H₂O at 3350 cm⁻¹. This approach is of particular use for mucoid bacteria.

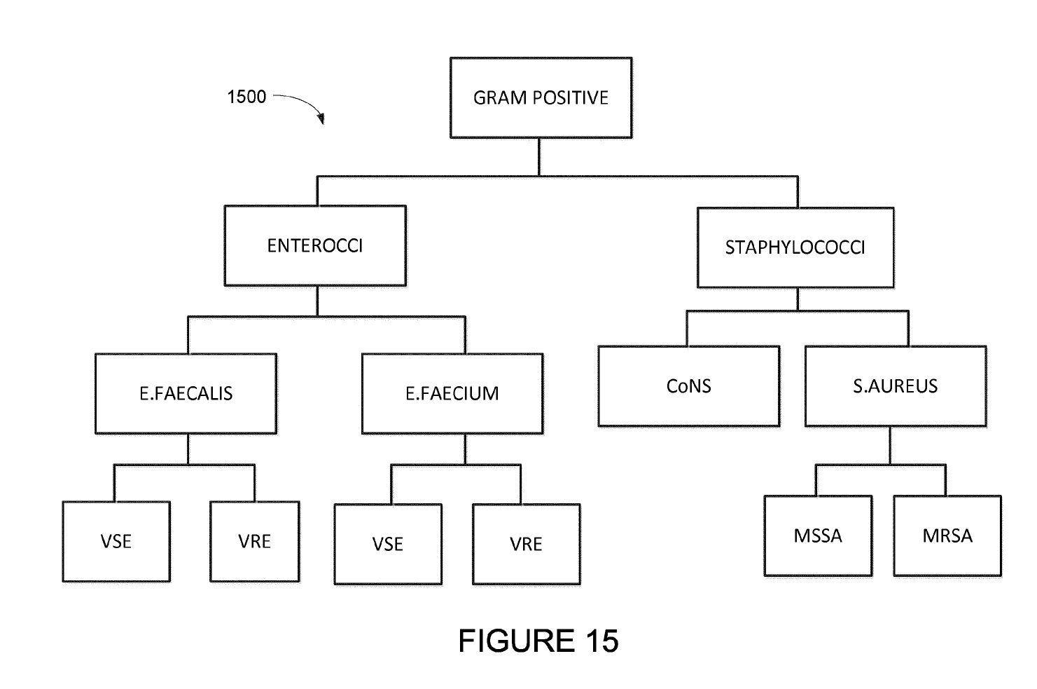

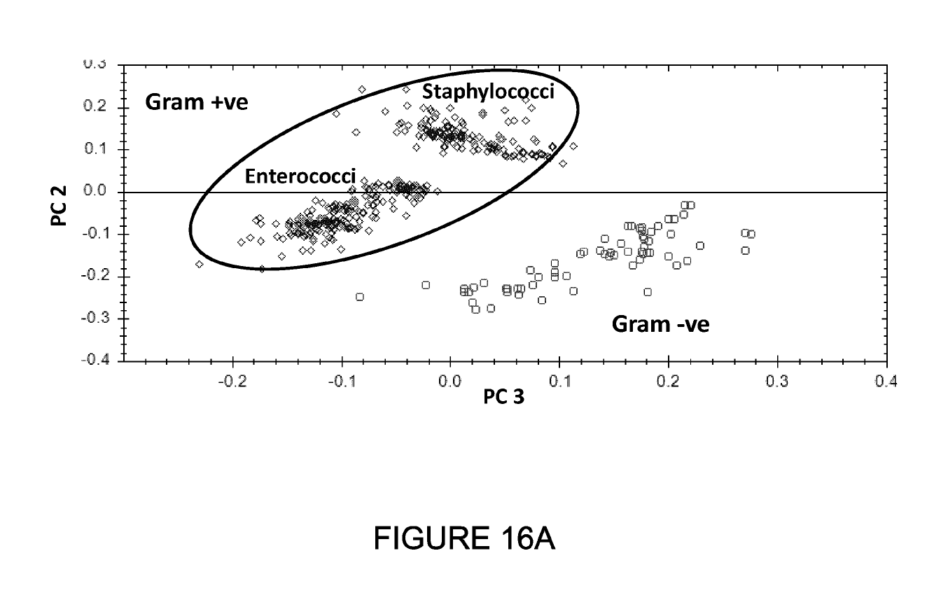

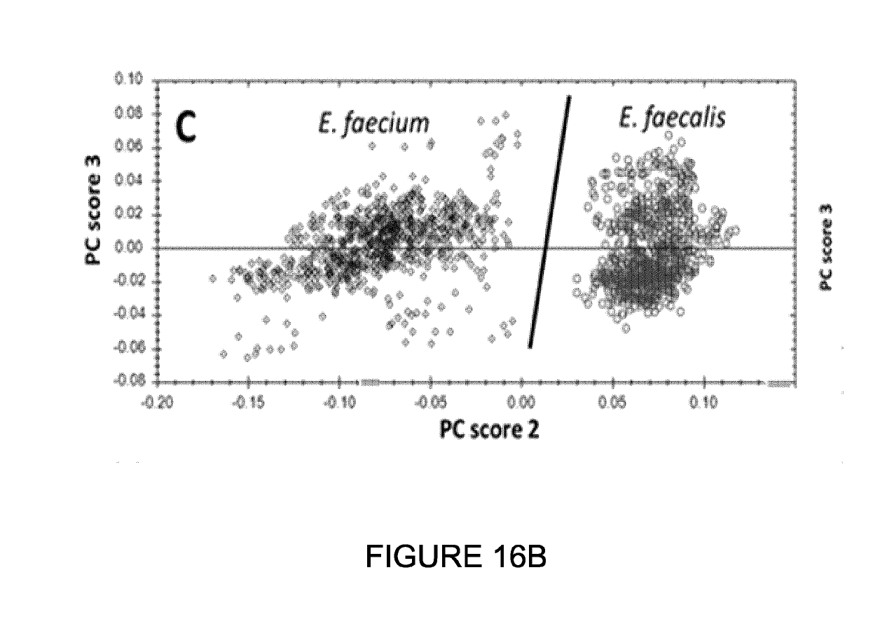

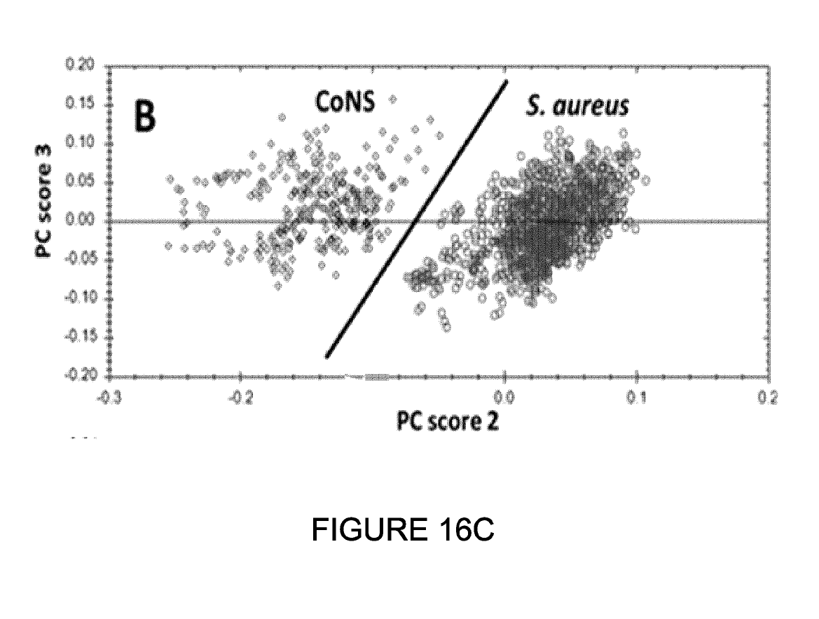

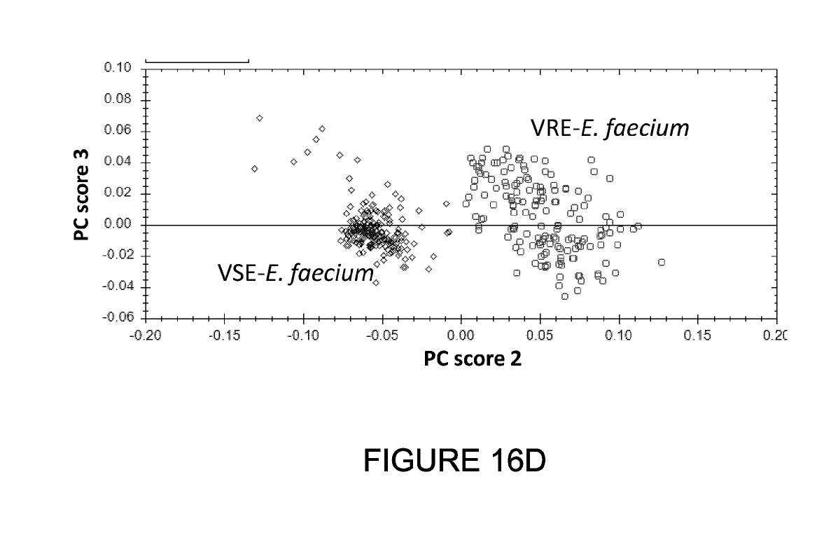

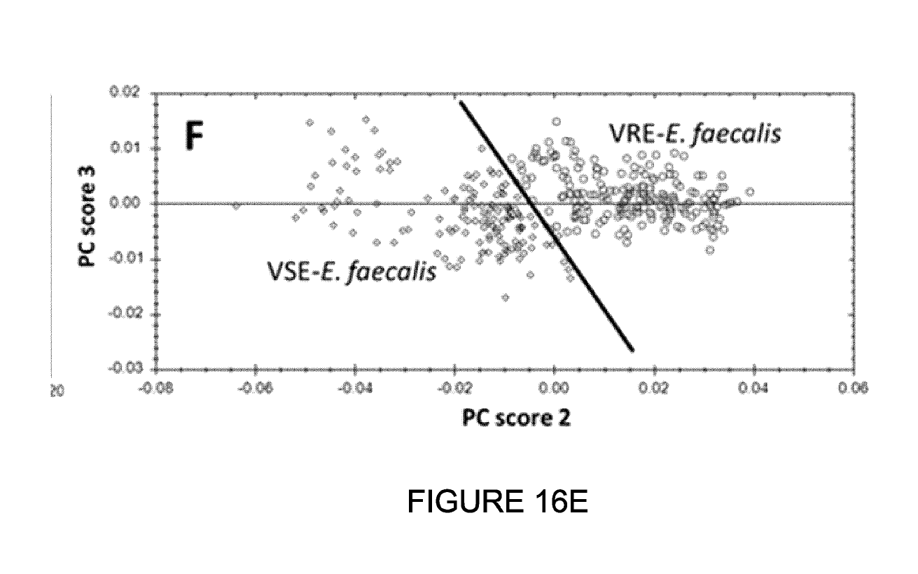

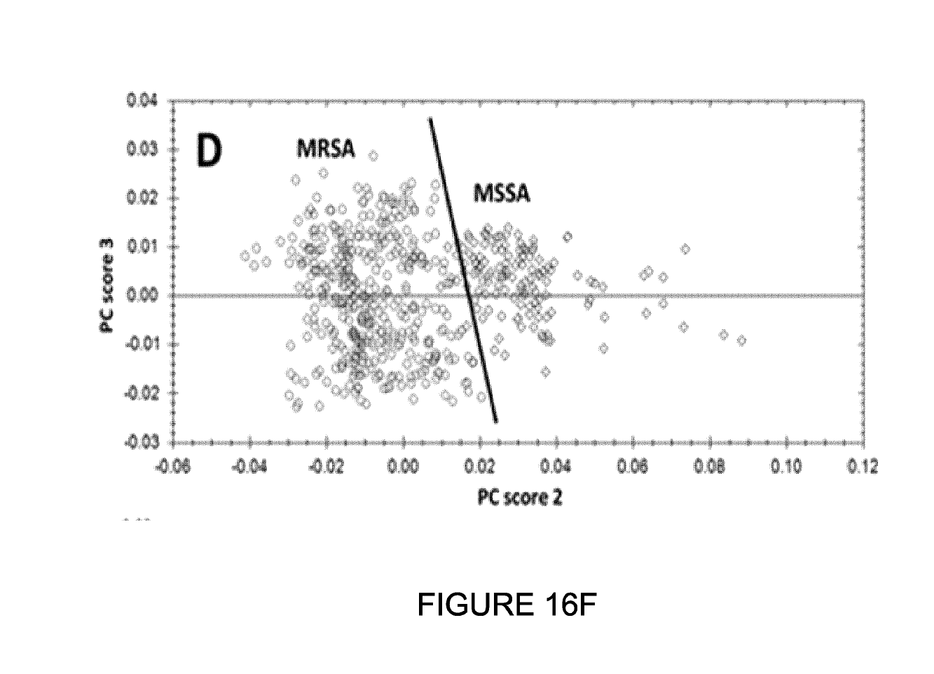

The method 200 may be used to discriminate between Gram-positive and Gram-negative bacteria by principal component analysis (PCA) of ATR-FTIR spectra. Thus far, it has been demonstrated that the method 200 has the capability to discriminate among nine (9) genera represented in an ATR-FTIR spectral database. An example database structure 1500 for Gram-positive bacteria is illustrated in FIG. 15. The top tier of the structure 1500 is Gram positive, followed by a second tier to discriminate between Enterococci and Staphylococci (see FIG. 16A). A third tier allows to discriminate between Enterococcus faecalis and E. faecium (see FIG. 16B) and between coagulase-negative staphylococci (CoNS (and Staphylococcus aureus (see FIG. 16C). In the fourth tier, the method 200 may thus be used to discriminate between vancomycin-resistant enterococci (VRE) and vancomycin-sensitive enterococci (VSE) (see FIGS. 16D and 16E), and to discriminate between methicillin-resistant Staphylococcus aureus (MRSA) and methicillin-sensitive Staphylococcus aureus (MSSA) (see FIG. 16F).

In the creation of a spectral database, the microorganisms may be cultured twice to ensure purity. Isolated colonies with the same morphology are selected and transferred to the surface of the internal reflection element for ATR-FTIR spectroscopic measurement. The ATR-FTIR spectrum is recorded. Replicate spectra may be obtained and those with the smallest standard deviation from the mean, are added to the database. Additional information may be added to a spectral file header, such as genus, species, strain, antimicrobial profile, growth medium, growth conditions, date, and the like.

In some embodiments, the modified spectral data is compared with spectral data of reference microorganisms obtained using a same culture medium as the sample. The use of another culture medium may result in an altered spectral profile. Therefore, the same media may be used to ensure that the same spectral profile is obtained. Alternatively, spectral data of reference microorganisms are obtained using a plurality of different culture media, and data from each spectral acquisition are pooled in order to make the reference data culture-media independent. In the example of FIG. 16A, the image corresponds to discrimination between Gram-positive and Gram-negative bacteria grown on two different culture media. Two or more media may be used to create a culture media-independent reference data set.

The method 200 may be used to identify antibiotic-resistant strains of microorganisms taken from culture media without the addition of any antibiotic. FIGS. 16D and 16E illustrate PCA scores plots showing clustering of clinical isolates of vancomycin-resistant enterococci (VRE) and vancomycin-sensitive enterococci (VSE) based on differences in their ATR-FTIR spectra following culture on 5% sheep blood agar.

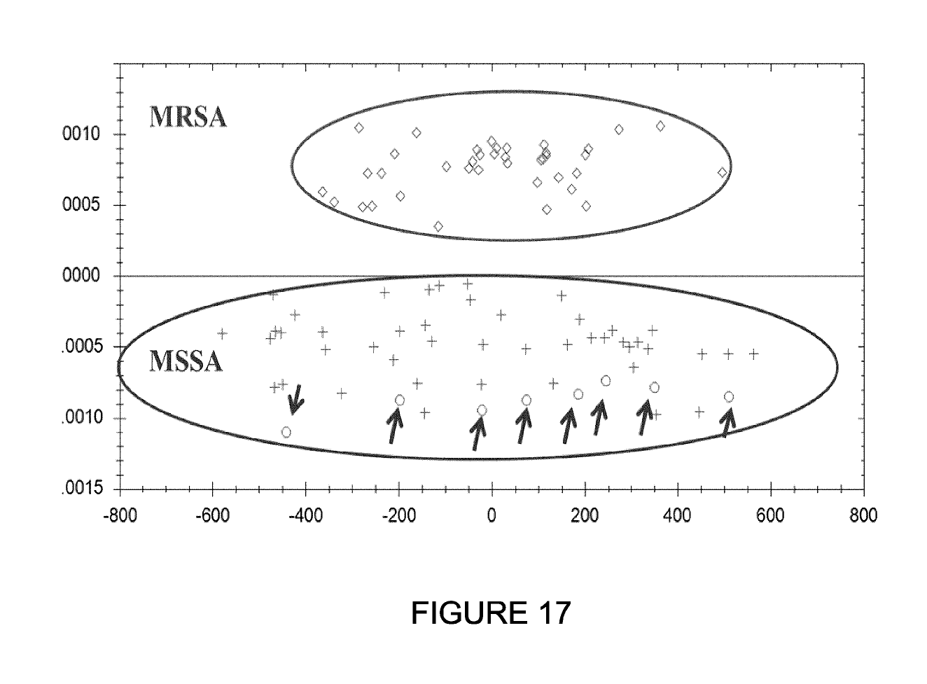

The method 200 may be used to identify microorganisms from positive blood cultures. While traces of blood in dried samples act as large contaminants, having the blood diluted in water causes the effect to be negligible. FIG. 17 illustrates a PCA scores plot showing clustering of methicillin-resistant Staphylococcus aureus (MRSA) and methicillin-susceptible Staphylococcus aureus (MSSA) clinical isolates based on differences in their ATR-FTIR spectra. The arrows indicate the spectra of bacteria separated from blood cultures. The remainder of the spectra were collected from microbial cultures.

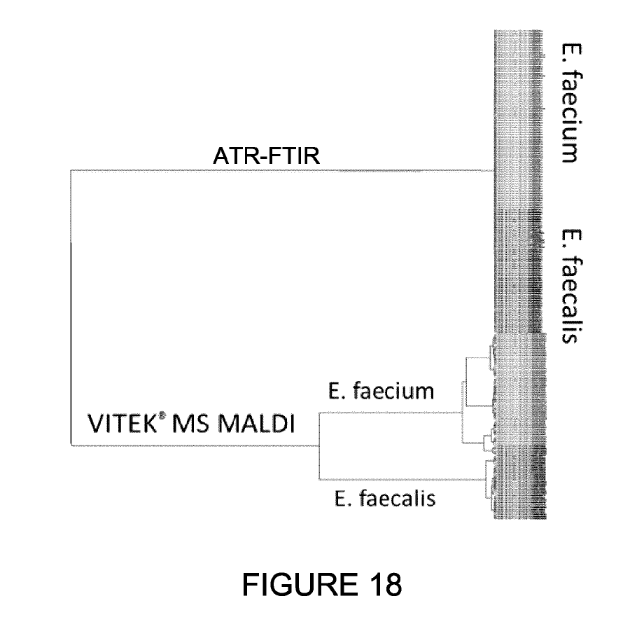

In some embodiments, infrared spectroscopy as described herein is used to enhance and/or refine characterization of a microorganism by matrix-assisted laser desorption-ionization time-of-flight (MALDI-TOF) mass spectrometry (MS). Referring to FIG. 18, there is illustrated an example of differentiation between Enterococcus faecalis and E. faecium by tethered spectral analysis (TSA). ATR-FTIR spectroscopy is used to find the spectral features (in the range of 1480-980 cm⁻¹) that differentiate the two microbial species. Extended-mass MALDI-TOF MS spectra (200-100,000 Da) are acquired of the same isolates. The two spectral data sets are then integrated for TSA by multivariate techniques or cross-correlation heterospectroscopy.

In some embodiments, the m/z data is combined with the ATR-FTIR data into a single stitched spectrum and TSA is carried out to identify the mass spectral and infrared spectral features that maximize the differentiation between two types of microorganisms.

In some embodiments, the prediction of an unknown microorganism is carried out by ATR-FTIR spectral analysis independent to the MALDI-TOF MS analysis. The identification of the unknown microorganism by the two independent means can further enhance the reliability of the identification by MALDI-TOF MS.

In some embodiments, other spectral data is acquired from another spectroscopic technique—such as ¹H (proton), ¹³C, ³¹P or ¹⁵N\nuclear magnetic resonance (NMR) spectroscopy, including solid-state high-resolution magic angle spinning (HRMAS) NMR. The ATR-FTIR data may thus be used to identify the spectral features responsible for the differentiation between two types of microorganisms. Subsequently or in tandem, other spectral data from other spectroscopic techniques can be utilized to identify the biomarker(s) associated with the infrared spectral features. In some embodiments, spectra generated from stitching of multiple spectral data sets from the above mentioned techniques can be subjected to TSA after spectral pre-processing, including normalization. Individually or combined, these methods increase the reliability of microbial identification by multispectral domain spectroscopy.

An example protocol for the separation of bacteria from blood culture broth for the purpose of identification by ATR-FTIR spectroscopy is as follows.

1. Aliquots from positive blood cultures are syringed into BD Vacutainer® SST™ serum separator tubes.
2. Tubes are centrifuged at 3,000×g.
3. The supernatant is removed and replaced with equal volume of saline.
4. Tubes are centrifuged at 3,000×g.
5. The supernatant is removed and replaced with 1 mL of saline to re-suspend the bacteria and the suspension is transferred to an Eppendorf tube.
6. Eppendorf tubes are centrifuged at 13,000×g (3 minutes).
7. Supernatant is decanted.
8. Eppendorf tubes are centrifuged at 13,000×g (10 minutes).
9. Residual supernatant is removed by using a cotton swab and bacteria are transferred to the ATR-FTIR surface by using a plastic applicator.

It should be noted that the sample may have been previously treated using various processes, such as those associated with clinical samples, subcultures, and/or frozen samples.

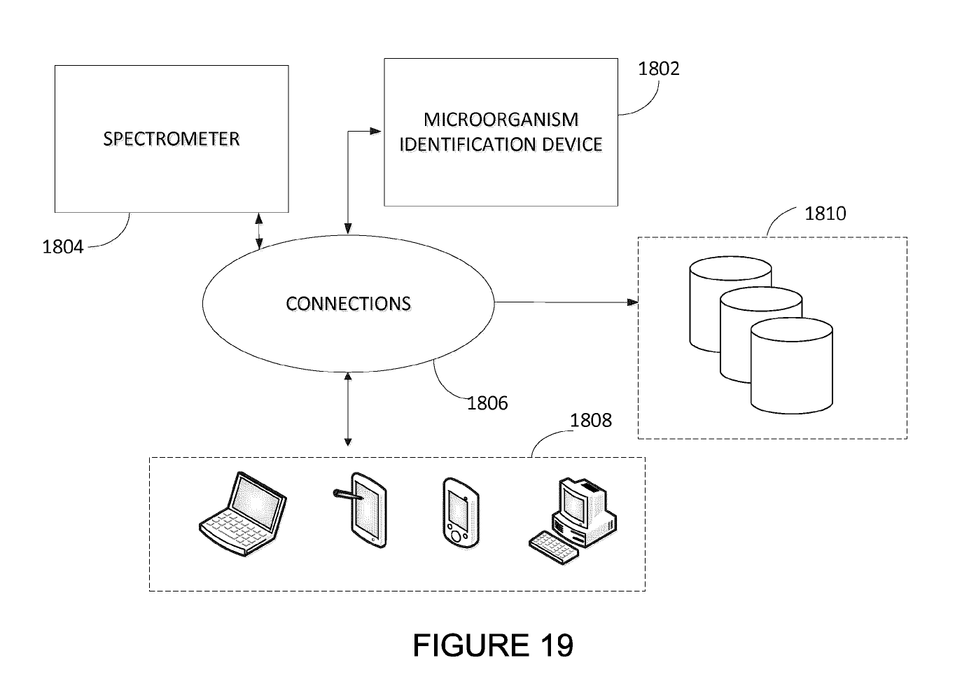

Referring to FIGS. 19 to 21, a system for spectral identification of microorganisms will now be described. In FIG. 19, there is illustrated a microorganism identification device 1802 operatively connected to spectrometer 1804. The microorganism identification device 1802 may be provided separately from or incorporated within the spectrometer 1804. For example, the microorganism identification device 1802 may be integrated with the spectrometer 1804, either as a downloaded software application, a firmware application, or a combination thereof. The spectrometer 1804 may be any instrument capable of acquiring infrared spectral data from an object, such as but not limited to an FTIR spectrometer. Some example spectral acquisition parameters are as follows:

Resolution: 1-64 cm⁻¹
Order of zero filling: 0-4
Detector type: DTGS. MCT, or other IR responsive detectors
Detector gain: 1-8
Apodization: triangular, Happ-Genzel, or boxcar
Number of scans=1-512
Spectral acquisition time: 1 second to minutes
Background (ideally before each sample: 1-512 scans
SNR: >10:1 (1380-980 cm⁻¹) (100% line) with residual water vapor<0.001 absorbance units

In some embodiments, the following protocol may be used for acquiring the background spectrum and spectral data with the spectrometer 1804.

1. Turning on the instrument and letting it warm up.
2. Launching the software on the computer and setting the spectral acquisition parameters to:
Number of scans: 128 scans (or another value, as desired)
Resolution: 8 cm⁻¹
   3. Cleaning the surface of the internal reflection element (such as a diamond ATR crystal) by gently rubbing it with a Kimwipe that has been wetted with 75% ethanol; and drying the surface with a clean Kimwipe.
   4. Collecting a background spectrum (noting that the surface of the internal reflection element must be bare, clean & dry).
   5. Collecting a small amount of bacteria (˜1-5 colonies) from a culture plate using a sterilized toothpick or loop without breaking the culture medium surface.
   6. Spreading the collected bacteria on the surface of the internal reflection element and ensuring that the entire surface is covered (˜2-mm in diameter).
   7. Pressing “Scan sample” to collect the spectral data.
   8. Cleaning the crystal by wetting the bacteria with a disinfecting fluid (75% ethanol or bleach)
   9. Wiping the bacteria off using a Kimwipe.
   10. Repeating steps 3 through 9 for each subsequent sample and acquiring a) a spectrum of water after every 20 samples to check the quality of the spectra, and b) a spectrum of an E. coli reference strain after every 30 samples. These numbers are purely illustrative and may be varied.
   11. Cleaning the sampling surface by the procedure in step 3 and turning off the instrument

The following experimental protocol was used for ATR-FTIR spectral acquisition. Gram-positive isolates were sub-cultured on 5% sheep's blood agar for 18-24 h at 35° C. With certain exceptions, Gram-negative isolates were sub-cultured on 5% sheep's blood agar or MacConkey agar for 18-24 h at 35° C. Following incubation, 2-3 isolated colonies were collected from the agar surface and spread on the sampling surface (2-mm diamond crystal) of the ATR-FTIR spectrometer and a spectrum was immediately recorded using a spectral acquisition time of 30 seconds. For each culture plate, 3-4 replicate spectra were acquired from different colonies.

Referring back to FIG. 19, various types of connections 1806 may be provided to allow the microorganism identification device 1802 to communicate with the spectrometer 1804. For example, the connections 1806 may comprise wire-based technology, such as electrical wires or cables, and/or optical fibers. The connections 1806 may also be wireless, such as RF, infrared, Wi-Fi, Bluetooth, and others. Connections 1806 may therefore comprise a network, such as the Internet, the Public Switch Telephone Network (PSTN), a cellular network, or others known to those skilled in the art. Communication over the network may occur using any known communication protocols that enable devices within a computer network to exchange information. Examples of protocols are as follows: IP (Internet Protocol), UDP (User Datagram Protocol), TCP (Transmission Control Protocol), DHCP (Dynamic Host Configuration Protocol), HTTP (Hypertext Transfer Protocol), FTP (File Transfer Protocol), Telnet (Telnet Remote Protocol), SSH (Secure Shell Remote Protocol), and Ethernet. The connections 1806 may also use various encryption means to protect any of the data acquired and/or transferred.

The microorganism identification device 1802 may be accessible remotely from any one of a plurality of devices 1808 over connections 1806. The devices 1808 may comprise any device, such as a personal computer, a tablet, a smart phone, or the like, which is configured to communicate over the connections 1806. In some embodiments, the microorganism identification device 1802 may itself be provided directly on one of the devices 1808, either as a downloaded software application, a firmware application, or a combination thereof.

One or more databases 1810 may be integrated directly into the microorganism identification device 1802 or any one of the devices 1808, or may be provided separately therefrom (as illustrated). In the case of a remote access to the databases 1810, access may occur via connections 1806 taking the form of any type of network, as indicated above. The various databases 1810 described herein may be provided as collections of data or information organized for rapid search and retrieval by a computer. The databases 1810 may be structured to facilitate storage, retrieval, modification, and deletion of data in conjunction with various data-processing operations. The databases 1810 may be any organization of data on a data storage medium, such as one or more servers or long-term data storage devices. The databases 1810 illustratively have stored therein spectral data for reference microorganisms used for comparison with spectral data of unknown samples.

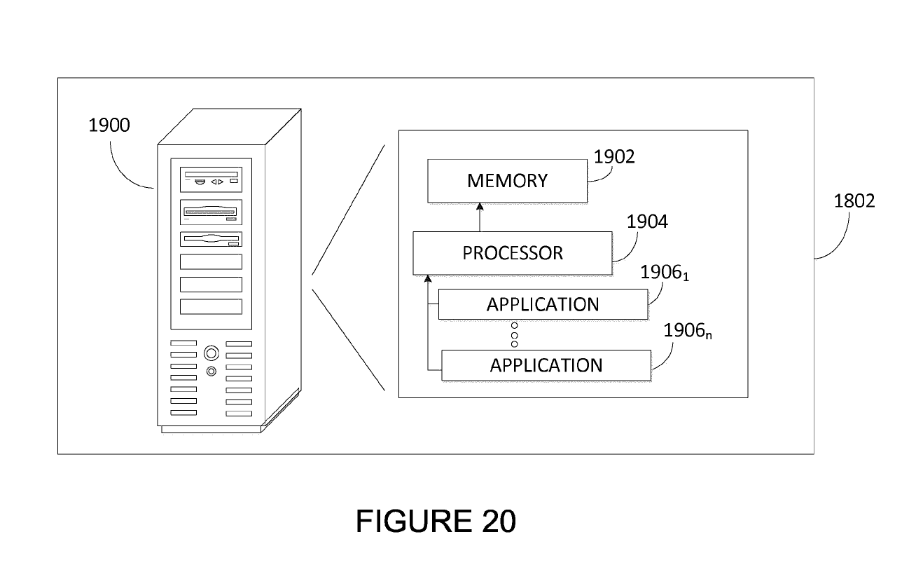

As shown in FIG. 20, the microorganism identification device 1802 illustratively comprises one or more servers 1900. For example, a series of servers corresponding to a web server, an application server, and a database server may be used. These servers are all represented by server 1900 in FIG. 20. The server 1900 may be accessed by a user, such as a technician or laboratory worker, using one of the devices 1808, or directly on the system 1802 via a graphical user interface. The server 1900 may comprise, amongst other things, a plurality of applications 1906₁ to 1906ₙ running on a processor 1904 coupled to a memory 1902. It should be understood that while the applications 1906₁ to 1906ₙ presented herein are illustrated and described as separate entities, they may be combined or separated in a variety of ways.

The memory 1902 accessible by the processor 1904 may receive and store data. The memory 1902 may be a main memory, such as a high-speed Random Access Memory (RAM), or an auxiliary storage unit, such as a hard disk, a floppy disk, or a magnetic tape drive. The memory 1902 may be any other type of memory, such as a Read-Only Memory (ROM), or optical storage media such as a videodisc and a compact disc. The processor 1104 may access the memory 1902 to retrieve data. The processor 1904 may be any device that can perform operations on data. Examples are a central processing unit (CPU), a front-end processor, a microprocessor, and a network processor. The applications 1906₁ to 1906ₙ are coupled to the processor 1904 and configured to perform various tasks. An output may be transmitted to the devices 1808.

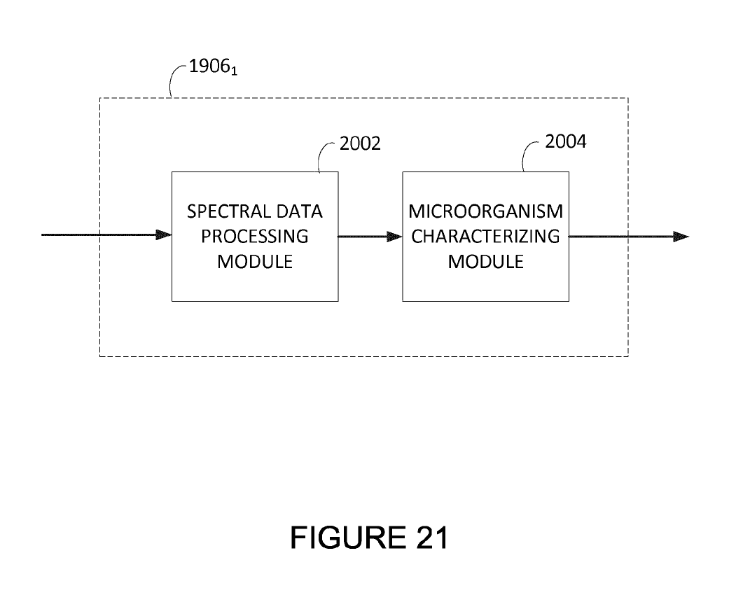

FIG. 21 is an exemplary embodiment of an application 1906₁ running on the processor 1904. The application 1906₁ illustratively comprises a spectral data processing module 2002 and a microorganism characterizing module 2004. The spectral data processing module 2002 is configured for receiving the background spectrum and the spectral data. The spectral data processing module 2002 may also be configured for combining the background spectrum and the spectral data to produce the modified spectral data. In some embodiments, the spectral data processing module is further configured for validating the modified spectral data, for example by comparing water vapor level, sample water content, and/or sample biomass to a threshold or a reference value. Some of the mathematical operations performed by the spectral data processing module 2002 on the background spectrum and/or spectral data include, but are not limited to, first derivatives, vector normalizations (4000-400 cm⁻¹), and cubic interpolation (with data spacing of 0.1-32).

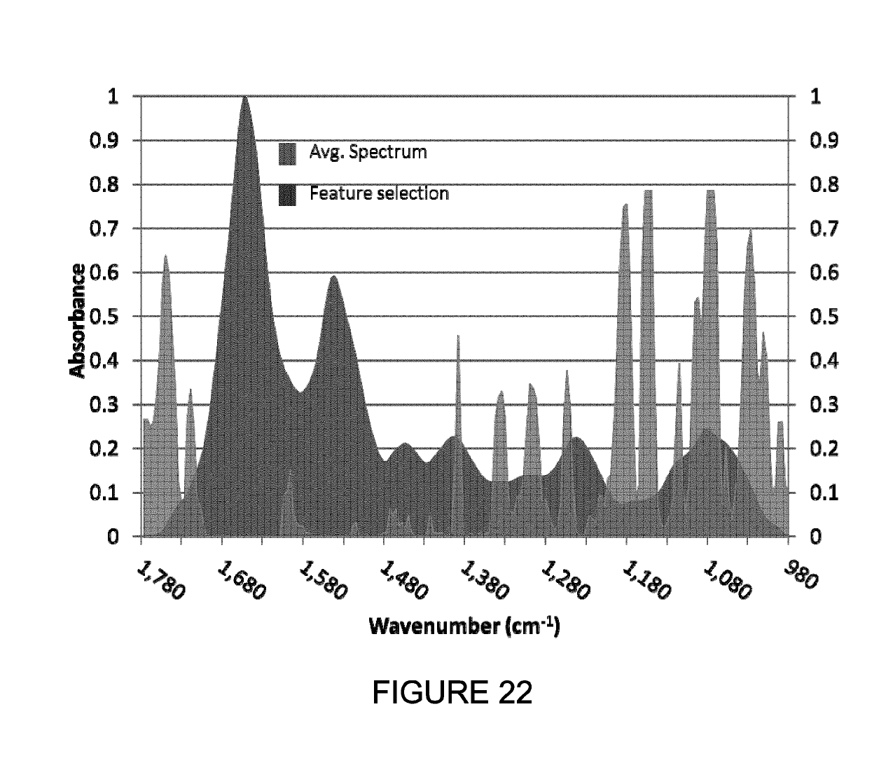

The microorganism characterizing module 2004 may be configured to receive the modified spectral data and to perform microorganism characterization by comparing the modified spectral data to reference spectral data of known microorganisms. In some embodiments, the microorganism characterizing module 2004 is configured to use target spectral regions in the modified spectral data pre-selected by applying a feature selection algorithm to training data as per U.S. Pat. No. 9,551,654. For example, a feature selection algorithm (FSA) is employed to identify the significant biochemical markers that are more relevant than the proteins in microbial identification. The comprehensive information content in the ATR-FTIR spectra can differentiate between types of bacteria at different levels of classification (genus, species, strain, serotype, and antimicrobial resistance characteristics and in some cases genotypic characteristics). Based on the FSA, spectral regions attributed to specific biomolecules can then be identified to increase the resolution power of MALDI-TOF MS in its ability to differentiate between closely related genera, such as E. coli and Shigella. FIG. 22 presents a comparison between an average spectrum and the spectral regions selected by using the FSA.

In some embodiments, a grid-greedy feature selection algorithm is used with three regions of a minimum size of 20 wavenumbers (6 features) and a maximum size of 92 wavenumbers (24 features) per region. All possible combinations of such regions are evaluated between 1780 and 980 cm⁻¹ and the region with the highest LOOCV-KNN classification score is selected. The greedy portion of the algorithm examines combinations of adjacent features following the path of greatest improvement. The forward selection begins by evaluating the single feature with the highest classification score, followed by adding features one at a time which keeps the score at a maximum. The routine stops when the classification score is no longer improved by adding features. The search may continue for a minimum of 21 features (10% of the total number of features) even if there is no further improvement in classification score in order to minimize over-fitting of the training data. Other feature selection algorithms may also be used.

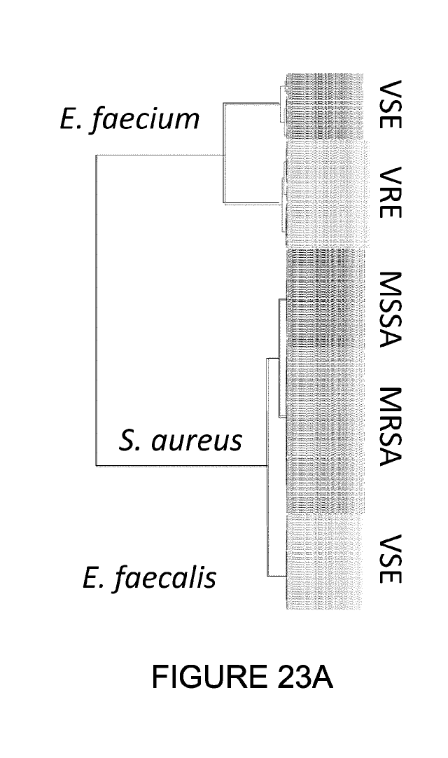

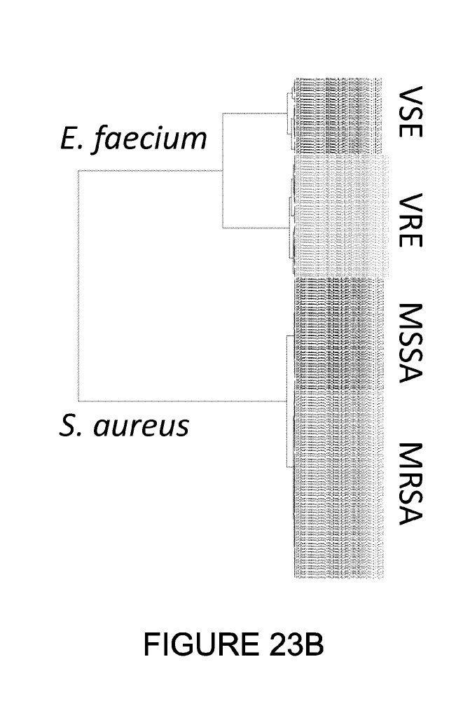

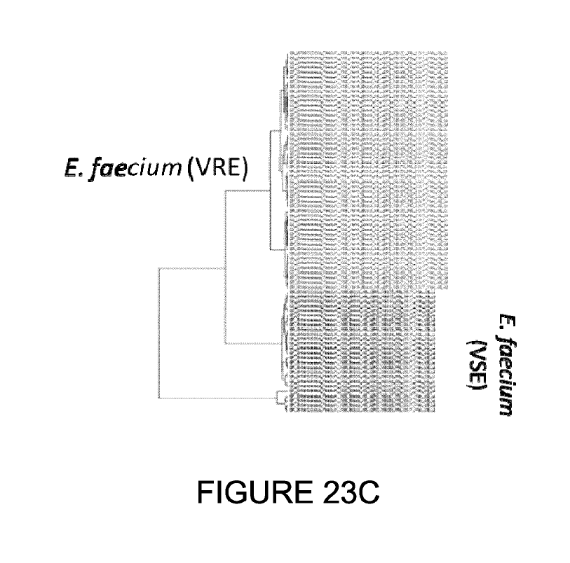

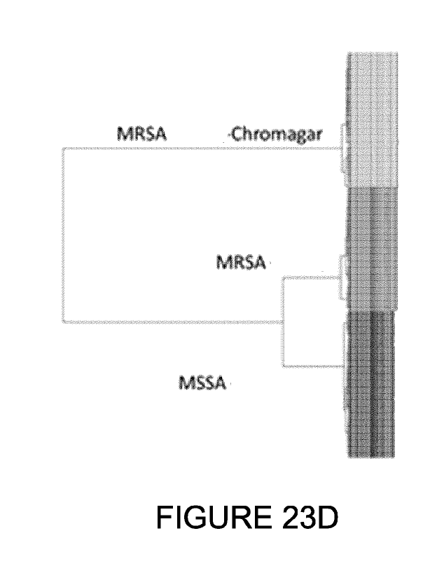

Additional examples are provided in FIGS. 23A-23D. FIG. 23A is an example of discrimination between microorganisms at different levels of classification. A differentiation was made among two genera, species, and antibiotic resistance characteristics simultaneously, employing FSA, yielding spectral regions of 980-991, 1030-1049, 1180-1196, 1234-1246, 1273-1296, and 1462-1473 cm⁻¹ to achieve optimal discrimination. Additional bands may be identified by adding more strains to the spectral database.

FIG. 23B is an example of discrimination between antibiotic-resistant and sensitive species by ATR-FTIR spectroscopy. Differentiation was made between two species belonging to different genera, with each having different antibiotic susceptibility profiles.

FIG. 23C is an example of the separation between antibiotic-resistant and sensitive strains by ATR-FTIR spectroscopy using the spectral region between 450 and 650 cm⁻¹. This was achieved using a full diamond internal reflection element 104 with no substrate. Spectral absorptions in this spectral region were found to be useful for the differentiation between vancomycin-resistant (VRE) and sensitive (VSE) strains of Enterococcus faecium. FSA analysis provided distinct spectral regions of 513-532, 567-575, 582-598, and 606-625 cm⁻¹.

FIG. 23D is an example of the separation between antibiotic sensitive and resistant microorganisms by ATR-FTIR spectroscopy. Based on the use of FSA, spectral regions were identified to separate MRSA from MSSA. This separation may be further enhanced by employing a growth medium with a defined concentration of antibiotic. Agar containing one or more antibiotics may be prepared in accordance with well established guidelines. Such agar plates may also contain a chromogenic compound that changes color during the growth of the microorganism and facilitates confirmation of antibiotic resistance. The use of the methods described herein obviates the need for chromogenic compounds as the ATR-FTIR spectrum of the microorganism will confirm the antibiotic susceptibility or resistance of the microorganism. Accordingly, no color change is required.

The methods and systems described herein employ a simple and universally applicable protocol that requires minimal sample preparation and no reagent beyond a culturing step. The method may be used with a high degree of automation and is amenable to micro colony analysis. It produces a fast turnaround time at a low cost per test, and is capable of detecting biochemical differences between antibiotic-resistant and susceptible bacterial strains in the absence of the antibiotic.

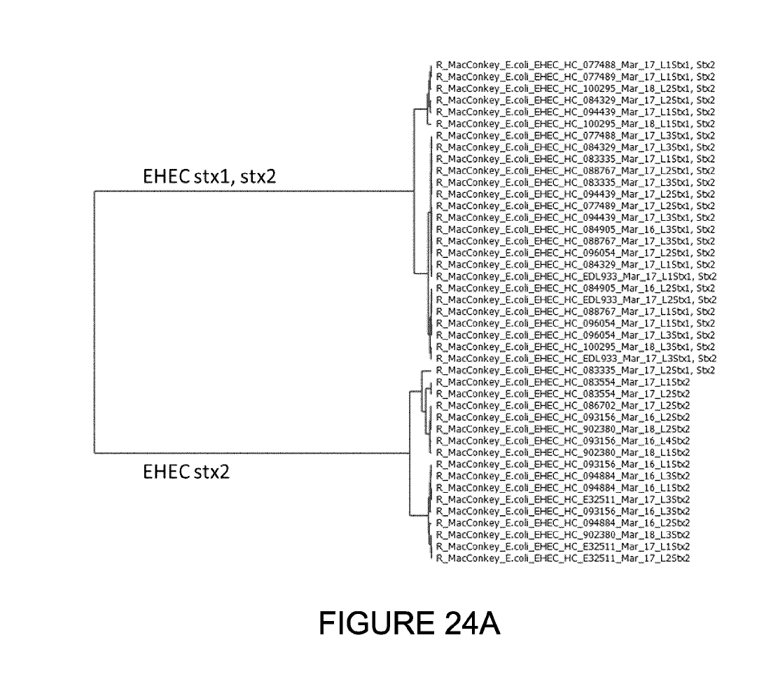

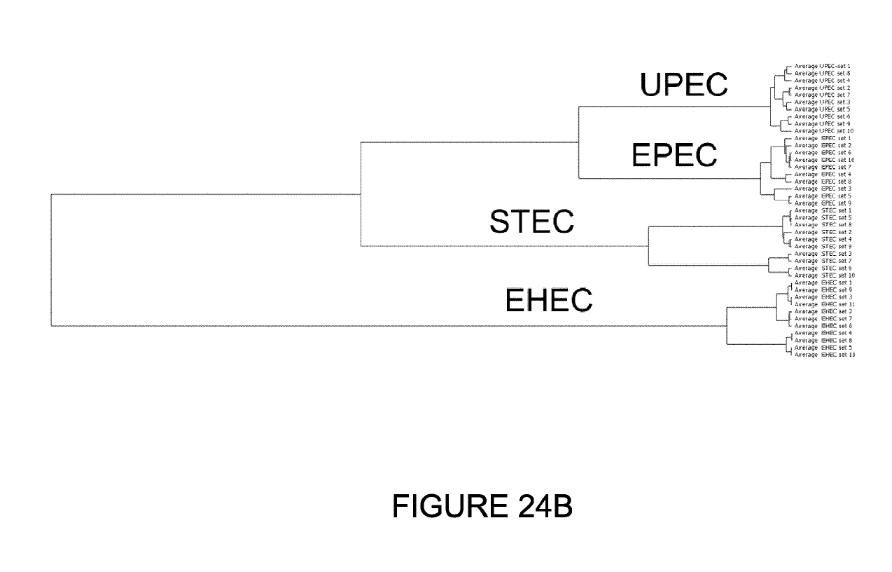

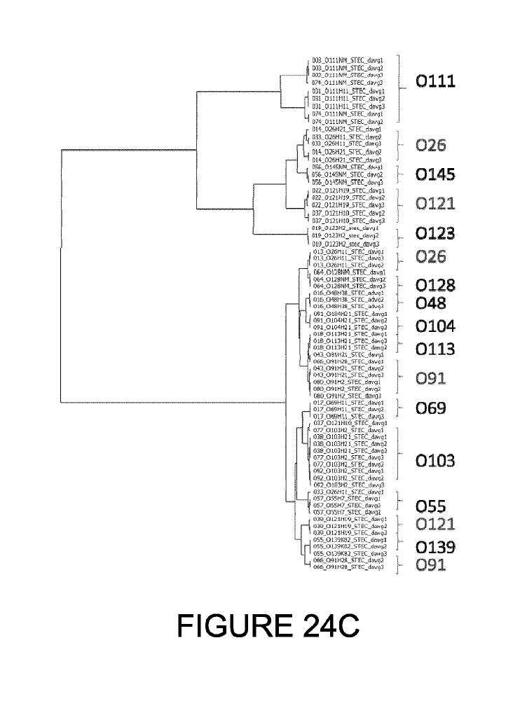

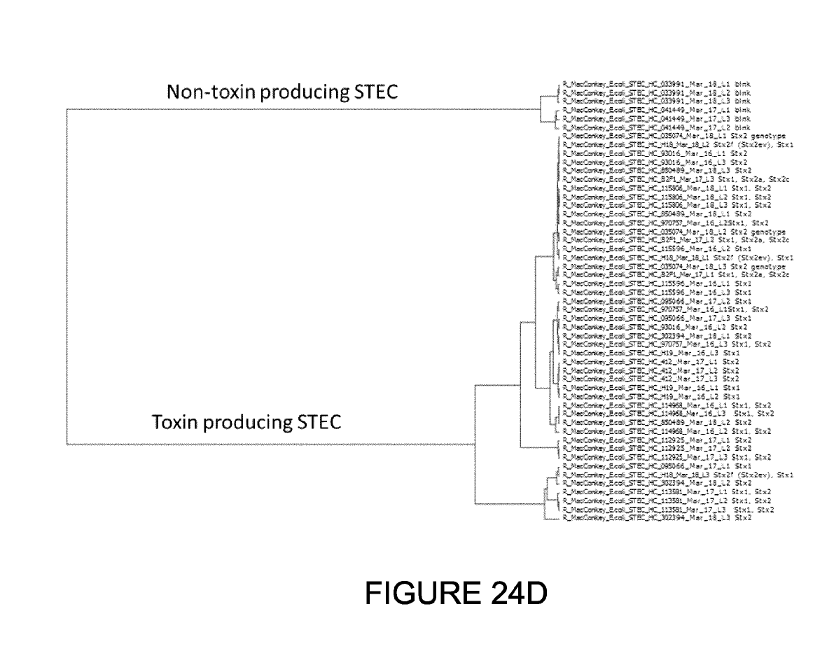

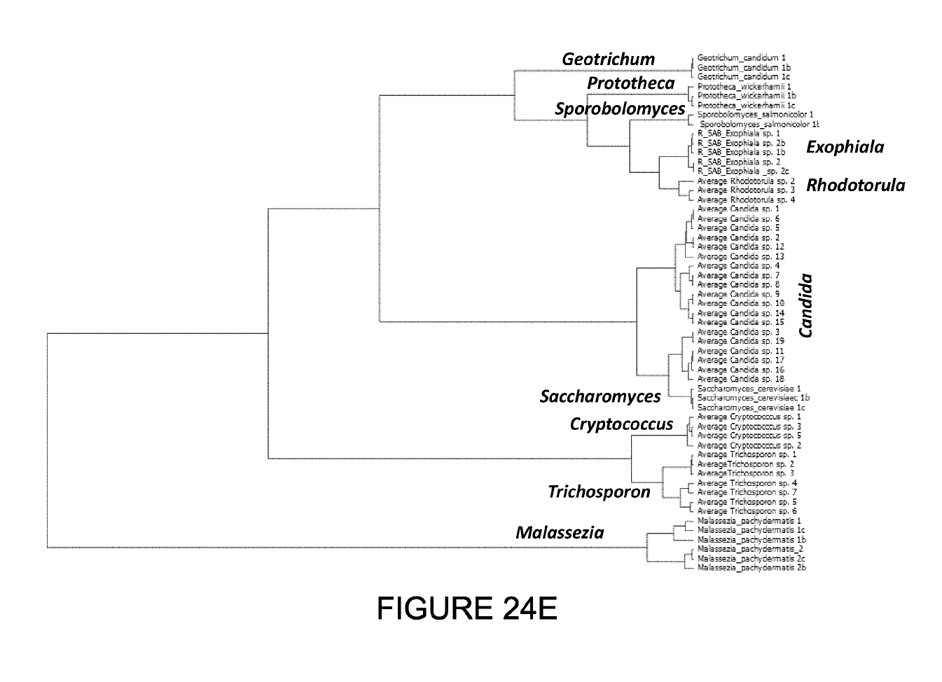

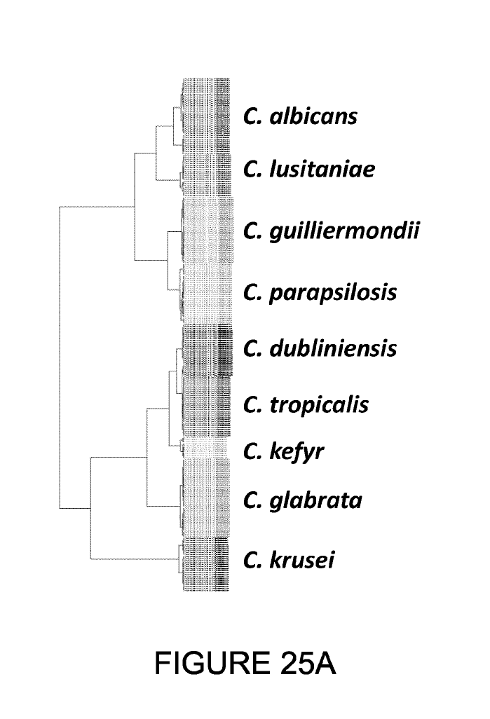

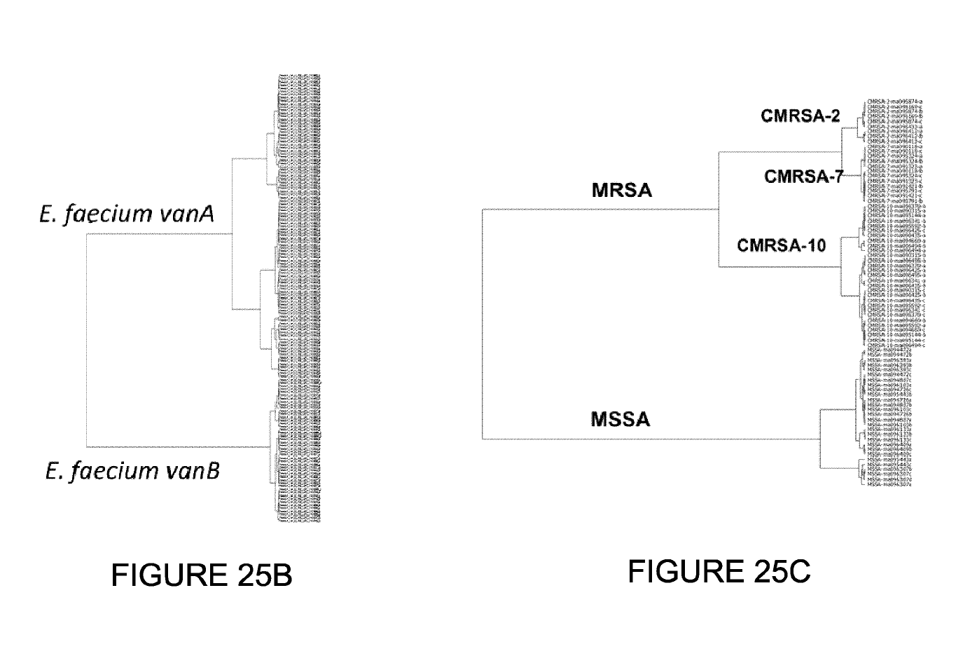

In addition to the above, the methods and systems described herein may be used, for example, for discriminating between microorganisms with varying resistance genes, differentiating between E. coli strains producing different toxins (see FIG. 24A), differentiating between E. coli pathotypes (see FIG. 24B), serotyping of E. coli pathotypes (see FIG. 24C), differentiating between toxin-producing and non-toxin-producing E. coli strains (see FIG. 24D), and discriminating among multiple genera of yeasts (see FIG. 24E). FIG. 25A illustrates an example for the discrimination among nine yeast species using the ATR-FTIR spectral data. FIG. 25B shows an example of discriminating between vanA and vanB genotypes of vancomycin-resistant Enterococcus faecium. FIG. 25C is an example of differentiation among methicillin-sensitive Staphylococcus aureus (MSSA) and three Canadian epidemic clones of methicillin-resistant S. aureus (CMRSA-2, CMRSA-7, and CMRSA-10).

The methods and systems described herein may also be used for the identification of clinical isolates from positive blood cultures. Indeed, as long as there is sufficient microorganism biomass that can be obtained from a positive blood culture, direct identification of bacteria may be performed using ATR-FTIR as described herein.

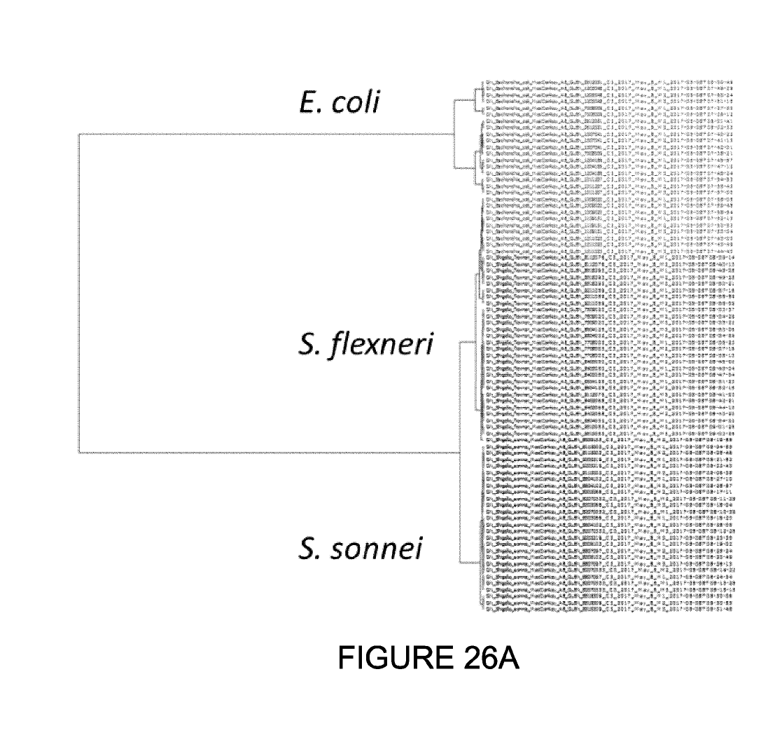

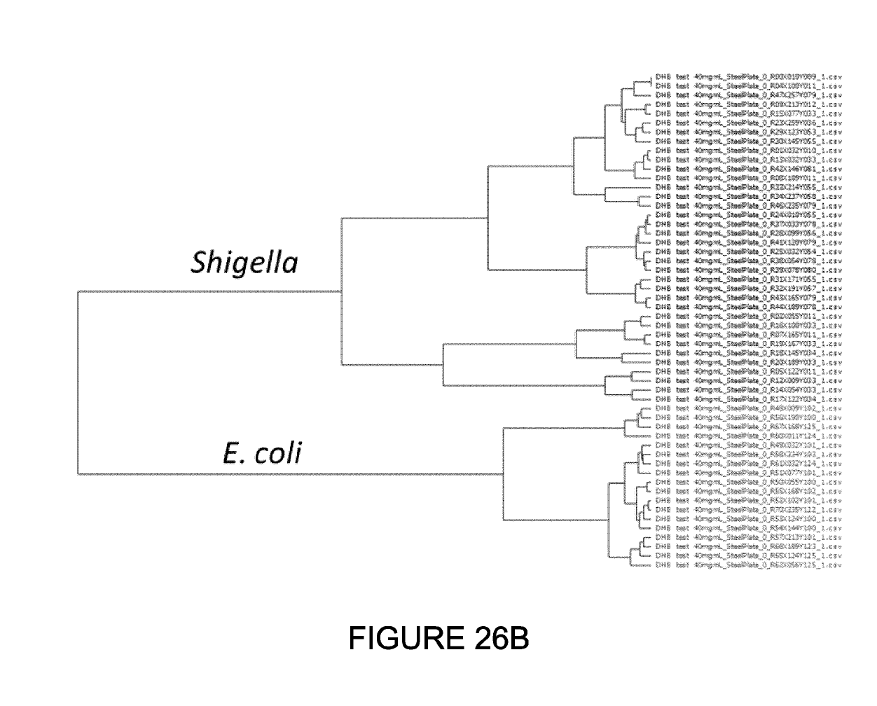

In some embodiments, the methods and systems described herein are used as a complementary technique to MALDI-TOF MS, for example to enhance the discrimination between MRSA and MSSA, VRE and VSE, and E. coli and Shigella spp. The methods and systems may also be used for the identification of Shiga-toxin-producing E. coli (STEC), the identification of non-fermenting Gram-negative bacilli isolated from patients with cystic fibrosis, and identification of yeasts. An example is illustrated in FIGS. 26A and 26B. FIG. 26A shows the differentiation between E. coli and Shigella species by ATR-FTIR analysis based on spectral regions in which non-protein biomolecules (i.e. phospholipids, nucleic acids, and carbohydrates) absorb infrared energy. The results of the ATR-FTIR analysis shown in FIG. 26A were used to guide the analysis subsequently performed with MALDI-TOF MS, illustrated in FIG. 26B, which shows differentiation between E. coli and Shigella species employing the m/z range of 600-1300.

In some embodiments, portable ATR-FTIR spectrometers may be used to perform the methods and implement the systems described herein.

The above description is meant to be exemplary only, and one skilled in the relevant arts will recognize that changes may be made to the embodiments described without departing from the scope of the invention disclosed. For example, the blocks and/or operations in the flowcharts and drawings described herein are for purposes of example only. There may be many variations to these blocks and/or operations without departing from the teachings of the present disclosure. For instance, the blocks may be performed in a differing order, or blocks may be added, deleted, or modified. While illustrated in the block diagrams as groups of discrete components communicating with each other via distinct data signal connections, it will be understood by those skilled in the art that the present embodiments are provided by a combination of hardware and software components, with some components being implemented by a given function or operation of a hardware or software system, and many of the data paths illustrated being implemented by data communication within a computer application or operating system. The structure illustrated is thus provided for efficiency of teaching the present embodiment. The present disclosure may be embodied in other specific forms without departing from the subject matter of the claims. Also, one skilled in the relevant arts will appreciate that while the systems, methods and computer readable mediums disclosed and shown herein may comprise a specific number of elements/components, the systems, methods and computer readable mediums may be modified to include additional or fewer of such elements/components. The present disclosure is also intended to cover and embrace all suitable changes in technology. Modifications which fall within the scope of the present invention will be apparent to those skilled in the art, in light of a review of this disclosure, and such modifications are intended to fall within the appended claims.

1. A method for spectral identification of a microorganism, the method comprising:
acquiring a background spectrum to measure a water vapor level of an ambient atmosphere in the absence of a sample;
bringing the sample containing the microorganism into contact with an internal reflection element in such a manner that water content of the microorganism is fully retained;
acquiring spectral data from the sample no more than a predetermined time after having acquired the background spectrum;
combining the background spectrum and the spectral data, thereby producing modified spectral data; and
characterizing the microorganism using the modified spectral data.

2. The method of claim 1, wherein the internal reflection element is a single-bounce attenuated total reflectance (ATR) element.

3. (canceled)

4. The method of claim 1, wherein the background spectrum is acquired in a path between an infrared source and an infrared detector defined for acquisition of the spectral data while the internal reflection element is without the sample.

5. (canceled)

6. (canceled)

7. The method of claim 1, wherein the predetermined time is less than or equal to 60 seconds.

8. The method of claim 1, wherein the predetermined time is comprised in a range of about two minutes to about five seconds.

9. (canceled)

10. (canceled)

11. (canceled)

12. (canceled)

13. The method of claim 1, wherein bringing the sample containing the microorganism into contact with the internal reflection element comprises providing the sample in a support that acts as a reservoir to retain the water content of the microorganism.

14. The method of claim 1, further comprising comparing the water vapor level of the modified spectral data to a first threshold and rejecting the modified spectral data when the water vapor level is above the first threshold.

15. The method of claim 1, further comprising comparing the water content level of the modified spectral data to a second threshold and rejecting the modified spectral data when the water content level is below the second threshold.

16. The method of claim 1, further comprising comparing a biomass of the sample, extracted from the modified spectral data, to a third threshold and rejecting the modified spectral data when the biomass is below the third threshold.

17. (canceled)

18. (canceled)

19. The method of claim 1, wherein acquiring spectral data from the sample comprises acquiring an attenuated total reflectance Fourier transform infrared spectrum.

20. The method of claim 19, further comprising using the modified spectral data to enhance the characterization of the microorganism by matrix-assisted laser desorption/ionization time of flight mass spectrometry.

21. A system for spectral identification of a microorganism, the system comprising:
a processing unit; and
a non-transitory computer-readable memory communicatively coupled to the processing unit and comprising computer-readable program instructions executable by the processing unit for:
receiving a background spectrum comprising a water vapor level of an ambient atmosphere;
receiving spectral data from a sample containing the microorganism and acquired no more than a predetermined time after the background spectrum is acquired, the microorganism in the sample having an intact water content;
combining the background spectrum and the spectral data, thereby producing modified spectral data; and
characterizing the microorganism using the modified spectral data.

22. The system of claim 21, wherein the predetermined time is less than or equal to 60 seconds.

23. The system of claim 21, wherein the predetermined time is comprised in a range of about two minutes to about five seconds.

24. The system of claim 21, wherein the program instructions are further executable for comparing the water vapor level of the modified spectral data to a first threshold and rejecting the modified spectral data when the water vapor level is above the first threshold.

25. The system of claim 21, wherein the program instructions are further executable for comparing the water content level of the modified spectral data to a second threshold and rejecting the modified spectral data when the water content level is below the second threshold.

26. The system of claim 21, wherein the program instructions are further executable for comparing a biomass of the sample, extracted from the modified spectral data, to a third threshold and rejecting the modified spectral data when the biomass is below the third threshold.

27. The system of claim 21, wherein characterizing the microorganism comprises comparing the modified spectral data to reference data to determine an identity of the microorganism.

28. (canceled)

29. The system of claim 21, wherein the program instructions are executable for performing attenuated total reflectance Fourier transform infrared spectroscopy.

30. The system of claim 29, wherein the program instructions are further executable for using the modified spectral data to enhance the characterization of the microorganism by matrix-assisted laser desorption/ionization time-of-flight mass spectrometry.
